(19) United States
(12) Patent Application Publication
(10) Pub. No.: US 2002/0004041 A1
(43) Pub. Date: Jan. 10, 2002

(54) Methods for abrogating a cellular immune response

(76) Inventors: Matthew L. Albert, New York, NY (US); Mithila Jegathesan, New York, NY (US); Robert B. Darnell, Pelham, NY (US)

Correspondence Address: KLAUBER & JACKSON, 411 HACKENSACK AVENUE, HACKENSACK, NJ, 07601

(21) Appl. No.: 09/804,584

(22) Filed: Mar. 12, 2001

Related U.S. Application Data
(63) Continuation-in-part of application No. 09/545,958, filed on Apr. 10, 2000.
(63) Continuation-in-part of application No. 09/251,896, filed on Feb. 19, 1999.

(51) Int. Cl. A61K 48/00; A61K 45/00; A61K 31/739; A61K 38/19

(52) U.S. Cl. 424/93.21; 424/93.7; 514/54; 424/85.1

(57) ABSTRACT

Methods are provided for preventing a cellular immune response to a pre-selected antigen by ex vivo or in vivo methods whereby dendritic cell maturation is permitted to occur in the absence of effective CD4+ T cell help. Under these conditions, elimination of cytotoxic T cells is achieved. The methods may be used for the prophylaxis of an undesired immune response to an autoimmune disease antigen, a transplant antigen, or reducing an exaggerated immune response to a antigen.

### CROSS-REFERENCE TO RELATED APPLICATIONS

This application is a continuation-in-part of U.S. Ser. No. 09/545,958, filed May 5, 2000, and a continuation-in-part of U.S. Ser. No. 09/251,896, filed Feb. 19, 1999, both of which are incorporated herein by reference in their entireties.

### GOVERNMENTAL SUPPORT

The research leading to the present invention was supported, at least in part, by a grant from the U.S. Public Health Service, National Institutes of Health, Grants No. GM-07793 and GM-55760. Accordingly, the Government may have certain rights in the invention.

### FIELD OF THE INVENTION

The invention in the field of immunology and relates to methods for preventing the development of a cellular immune response to a particular antigen, useful for the prophylaxis or treatment of autoimmune diseases, prevention of transplant rejection, or for reducing an inappropriately robust cellular immune response.

### BACKGROUND OF THE INVENTION

While central tolerance offers a mechanism for the deletion of potentially auto-reactive cytotoxic T lymphocytes (CTLs), additional strategies must be employed in order to account for the tolerization of T cells specific to tissue-restricted antigen (proteins uniquely expressed in peripheral tissues, e.g. cell-specific antigens; see J. F. Miller, G. Morahan, Annu Rev Immunol 10, 51-69, 1992). Experimental systems used to investigate peripheral tolerance have relied on adoptive transfer of mature naive CTLs isolated from T cell receptor (TCR) transgenic mice in which the TCR is specific for peptide epitopes derived from tissue-restricted antigens (C. Kurts, H. Kosaka, F. R. Carbone, J. F. Miller, W. R. Heath, J Exp Med 186, 239-45, 1997; A. J. Adler et al., J Exp Med 187, 1555-64, 1998; S. Webb, C. Morris, J. Sprent, Cell 63, 1249-56, 1990). T cells upregulate activation markers, undergo several rounds of cell division, after which they die a Fas-dependent apoptotic death (C. Kurts, H. Kosaka, F. R. Carbone, J. F. Miller, W. R. Heath, J Exp Med 186, 239-45,1997; C. Kurts, W. R. Heath, H. Kosaka, J. F. Miller, F. R. Carbone, J Exp Med 188, 415-20, 1998). Studies have also established that a bone-marrow-derived antigen presenting cells (APCs), and not the peripheral tissue itself, is responsible for the tolerization of antigen-specific CTL cells (C. Kurts et al., J Exp Med 184, 923-30, 1996). This indirect pathway for the inactivation of self-reactive CTLs has been termed ‘cross-tolerance’ (W. R. Heath, C. Kurts, J. F. Miller, F. R. Carbone, J Exp Med 187, 1549-53, 1998), as exogenous antigen must be cross-presented by the APC, resulting in the generation of MHC I/peptide complexes. While this work has established a new paradigm for understanding peripheral tolerance, the lack of an in vitro system to study cross-tolerance has prevented the precise definition of the cellular events responsible for this in vivo phenomenon. These include a failure to characterize (i) the mechanism of antigen transfer to the APC; (ii) the identification of the APC responsible for mediating this pathway; and (iii) the critical features which distinguish cross-priming from cross-tolerance.

Previous work has established that human dendritic cells (DCs) may acquire viral or tumor antigen from apoptotic cells in a manner which permits the formation of peptide/MHC I complexes and the activation of viral or tumor-specific CD8+ memory T cells, respectively (M. L. Albert, B. Sauter, N. Bhardwaj, Nature 392, 86-9, 1998; M. L. Albert et al., Nat Med 4, 13214, 1998; U.S. Serial Nos. 60/075,356; 60/077,095; 60/101,749; 09/251,896; PCT/US99/03763).

It is toward the development of a physiologically-relevant in-vitro system for cross-tolerance which accurately models the in vivo work of others, thus allowing the aforementioned unknowns to be addressed and to define the cellular mechanism underlying peripheral tolerance, as well as the identification of conditions that may be employed in vivo or ex vivo for skewing the immune system towards cross-tolerance, in order to abrogate or reduce a cellular immune response to a particular antigen, that the present invention is directed.

The citation of any reference herein should not be construed as an admission that such reference is available as “Prior Art” to the instant application.

### BRIEF SUMMARY OF THE INVENTION

The present invention is broadly directed to in-vivo and ex-vivo methods for reducing or preventing the development of a cellular immune response to a particular pre-selected antigen. Such prevention of the formation of effector (cytotoxic or killer) T-cells (CD8+ or CTLs) may take the form of inducing immunologic tolerance to the antigen. Immunologic tolerance may result in the deletion of naive or memory CD8+ T cells specific for a pre-selected antigen, or the skewing of an immune response such that no cytotoxic T cells capable of recognizing the antigen are functional. This latter example includes differentiating an immune response towards a Th2 response and inducing anergy of antigen specific T cells. As will be elaborated on in detail below, this immunologic outcome may be manipulated in vivo or ex vivo by carrying out the methods of the invention, following the processing of the desired antigen by dendritic cells and presentation of antigen-derived peptides in a complex with MHC I (also known as and interchangeably referred to as the histocompatability antigens, HLA-A,B,C). The inventors demonstrated that the activation of effector T cells via the cross-priming pathway requires the maturation of dendritic cells, and in addition, the participation of effective CD4+ T cell help. In defining the role of cross-presentation for the tolerization of T cells the inventors discovered by surprise that by permitting dendritic cell maturation while preventing effective CD4+ T cell help, immunologic tolerance results. The methods pertinent to the invention relate to the induction of immunologic tolerance, the conditions under which such tolerance may be achieved being heretofore unknown. Thus, the immune system may be manipulated in vivo or ex vivo (in vitro) to induce tolerance to an antigen.

The invention is also directed to an in-vitro model system in which tolerance to a pre-selected antigen is achieved. By use of this system, the importance of various components may be investigated, and the utility of compounds or agents that agonize or antagonize particular steps in the tolerizing pathway may be identified and optimized as potential agents for clinical utility. For example, agents such as antibodies to dendritic cell maturation markers, or to cytokines and their receptors whose interaction is required for the dendritic cell to receive effective CD4 T cell help, may all be evaluated. In addition, the role of inhibitors of signal transduction events triggered by CD4 T cell—dendritic cell engagement, or in absence of engagement, of extracellular signals with equivalent function, may be investigated.

The methods of the invention may be carried out ex vivo or in vivo. Dendritic cell maturation may be assured by permitting activity within the methods of the invention of agents which result in the upregulation of co-stimulatory molecules, such as but not limited to TNF, PGE2, LPS, CpG-DNA, which are required for inducing dendritic cell maturation. With regard to the elimination of effective CD4+help, in the methods of the invention, this takes the form of various means for either eliminating the CD4+ T cells themselves from the ex-vivo or local invivo environment; or intervening in the activity of one or more members of interacting, extracellular (secreted or cell surface) CD4⁺ T cell or dendritic cell products, such as the MHC II/peptide complex interaction with the CD4+T cell receptor, or a receptor or its ligand required for CD4/DC engagement and signaling; or by means of interfering with the intracellular signaling induced by the presence of the cells or the consequence of the interaction of the abovementioned extracellular products. In practice, such means include but are not limited to eliminating CD4+ T cells from an ex-vivo system or from the in-vivo site of immune activation, or preventing the consequences of interaction between CD4+ T helper cells and dendritic cells by interfering with the interaction between various receptor-ligand pairs known to be involved in CD4⁺ T cell/DC interactions. These include but are not limited to the MHC II/peptide complex, co-stimulatory molecules, adhesion molecules, or members of the TNF superfamily of receptor/ligand pairs. It also includes molecules able to substitute for CD4+ T cell help in the generation of CD8 effector cells, such as, by way of non-limiting example, CD40 ligand and CD40, TRANCE (also known as RANK ligand) and TRANCE receptor (also known as RANK), OX40 ligand and OX40, TWEAK and DR3 and interfering with other ligand-receptor interactions which abrogate the participation of effective CD4+ help on the development of a cellular immune response (i.e., T cell activation or priming). In addition, the downstream signal transduction pathways consequent to the interaction between the aforementioned receptor-ligand pairs are also effective targets for eliminating effective CD4+ help. Such may be achieved, for example, using compounds which antagonize FK binding protein (FKBP), such as FK-506, or compounds that antagonize TOR, such as rapamycin, either of which are also effective at achieving the desired tolerance. Finally, by inhibiting formation of mature forms of MHC II/peptide complexes within the dendritic cell by way of non-limiting example, preventing cleavage of invariant chain using cathepsin inhibitors, blocking loading of peptides by inhibiting HLA-DR, preventing successful antigen degradation and MHC II peptide epitope by inhibiting cathepsin D or alternative proteases, or by inhibiting transport of MHC II/peptide complexes to the cells surface. These various routes for assuring dendritic cell maturation and blocking effective CD4+ T cell help may be selected for the particular method undertaken to induce tolerance.

The methods of the invention are generally directed at preventing or obviating an unwanted immune response, such as treating a patient prior to transplant in order to obviate an immune response to the foreign antigens in the transplant. Transplant antigens include those donor antigens that are ‘allogeneic’ or ‘xenogeneic’ to the host. Transplant rejection is due to immune attack of the donor material; by tolerizing the host prior to, or during transplant, it may be possible to prevent, delay or treat active graft rejection. Autoimmune conditions in which a cellular immune response to a self antigen is responsible for pathology is another suitable use of the present methods. Self antigens to which tolerance is important include all antigens targeted during autoimmune disease (including but not limited to psoriasis, multiple sclerosis, type I diabetes, pemphigus vulgaris, rheumatoid arthritis and lupus).

Although current immunotherapy strategies to treat tumors are aimed at activating tumor-specific T cells, in some instances, autoimmunity has occurred. At such times, it would be useful to have strategies to interrupt this aberrant immune attack. The immune attack in response to some pathogens (e.g. mycobacteria, HIV), leads to wasting syndromes. In part, this is due to an excessive immune reaction due to the presence of a chronic infection. It may therefore be beneficial to dampen the immune response by partially tolerizing pathogen-specific T cells. Thus, suitable antigens for which tolerance is desirably induced by the methods of the invention include but are not limited to self antigens, transplant antigens, tumor antigens, and viral antigens, but these are merely illustrative and non-limiting.

In the methods for inducing tolerance to a pre-selected antigen, dendritic cell maturation is required together with inhibition of effective CD4+ help. In an example of the practice of the invention, tolerance to a pre-selected antigen may be induced either in vivo or ex vivo by providing a pre-selected antigen such that dendritic cells can process the antigen, mature, and present antigen-derived peptides in complexes with MHC I, for presentation to CD8⁺ T cells. Thus, in this aspect of the invention, signals permitting dendritic cell maturation and peptide presentation are necessary. In addition, effective CD4+ T cell help is blocked. For ex-vivo methods, in a non-limiting example, apoptotic cells expressing or containing the pre-selected antigen are exposed to dendritic cells derived from the patient, in the presence of maturation stimuli such as TNF, PGE2, etc. The ex-vivo system eliminates effective CD4+help by a means such as:

i) eliminating CD4+ cells from the ex-vivo system;

ii) inhibiting generation of MHC II peptide complex formation on the dendritic cell or preventing MHC II/peptide complex engagement with the CD4 T cell receptor;

iii) including CD4+cells in the ex-vivo system, but including at least one inhibitor of the interaction between a TNF superfamily member and its receptor; or

iv) including CD4+ cells in the ex-vivo system, but including an inhibitor of signal transduction from any one or more of the foregoing steps.

The four foregoing methods may be employed singly or in combination, depending on the purity of the cellular population, or other considerations such as the effectiveness of inhibiting a single receptor-ligand or signal transduction pathway member. In one embodiment, a combination of inhibitors of the interaction between various TNF superfamily members and their corresponding receptors is used. In a preferred embodiment, dendritic cells are treated with one or more of the aforementioned signal transduction inhibitors prior to re-infusion into the individual where CD4⁺ T cells exist. Any of the foregoing agents or combinations thereof is applied such that the DC receptors are prevented from engaging with antigen-specific CD4+ T cells; the signaling of the DC TNF superfamily receptors are blocked; and/or the generation of the MHC II/peptide complex is inhibited so that the DC can not engage the CD4⁺ T cell.

CD4+ cells may be eliminated from the ex-vivo system by including a purification step to remove CD4+ cells, or a cytotoxic CD4+ reagent such as antibodies to CD4 in combination with compliment may be used to treat isolated peripheral blood mononuclear cells before the exposure to antigen and the necessary reagents to assure dendritic cell maturation. If CD4 T cells are present in the ex-vivo system, or for in-vivo use, inhibiting the interaction between a TNF superfamily member and its receptor may be achieved using, for example, an antibody or antagonist of the binding of CD40 with its ligand, or with other TNF superfamily members and its receptor. Examples of such reagents include blocking antibodies, receptor decoys, or small molecule inhibitors, used singly or in combination. Preferably used are membrane-permeable compounds that inhibit signal transduction downstream from one of the foregoing steps. For example, interfering with FKBP activity or with TOR activity is a route to achieve the desired outcome herein. Such may be achieved by the use in the ex-vivo system by using FK-506, or rapamycin, respectively. These are merely non-limiting examples of agents with the desired activities which may be used effectively to achieve the desired tolerance of the immune system to the pre-selected antigen.

Following the above steps, the cellular components of the ex-vivo system may be introduced into the patient. As will be seen below, cells treated as above result in the deletion of antigen-specific CD8+ cells.

Various alternate steps may be performed which achieve the desired outcome and are fully embraced herein. For example, the antigen may be provided in the form of apoptotic cells expressing the antigen, or apoptotic cells loaded with the antigen. Other exogenous routes of antigen delivery are embraced herein. The dendritic cells may be derived from the patient, or an HLA-matched cell line may be used, such as an artificial antigen presenting cell (APC). As noted above, depending on the effectiveness of each of these means to reduce or eliminate effective CD4+ help in the system, various combinations of methods may be employed, such as partial elimination of CD4+ helper T cells, use of antibody against TRANCE, CD40, OX40, DR3, and the use of a signal transduction inhibitor such as FK-506 or rapamycin.

In the practice of the invention in vivo, temporary localization of the cellular components is desirable. For example, dendritic cells may be attracted to a particular intradermal or subcutaneous site in the body by placement on the skin of a transcutaneous delivery device comprising a dendritic cell chemoattractant. The delivery device also delivers a pre-selected antigen, as well as a blocker of effective CD4+ help, such as an FKBP or TOR antagonist, by way of non-limiting example, FK506 or rap amycin, respectively. Dendritic cells having encountered antigen at the intradermal or subcutaneous site, in the absence of effective CD4+ help, will proceed to induce tolerance of antigen-specific CD8+ T cells, resulting in immune tolerance to the antigen.

It is therefore an object of the invention to induce immunologic tolerance by cross-presenting antigen in the presence of a dendritic cell maturation stimulus but in the absence of effective CD4+ help.

It is another object of the present invention to provide a method for inducing apoptosis in antigen-specific cross-primed CD8+ cells in order to tolerize a mammalian immune system to the antigen by exposing dendritic cells to the antigen in the presence of a dendritic cell maturation stimulus and in the absence of effective CD4+ help.

It is yet a further object of the invention to inhibit the ability of a dendritic cell from activating antigen-specific CD8+ cells after cross-presentation of antigen by either inhibiting dendritic cell maturation or inhibiting effective CD4+ help.

These and other aspects of the present invention will be better appreciated by reference to the following drawings and Detailed Description.

### BRIEF DESCRIPTION OF THE DRAWINGS

FIGS. 1A-D demonstrate that CD4⁺ T cell help is required for the activation of CD8⁺ T cells and the production of IFN-γ.

FIGS. 2A-B show that TRANCE and CD40L substitute for CD4 help.

FIGS. 3A-B show that soluble lymphokines facilitate the cross-priming of CD8⁺ T cells.

FIS. 4A-B show that CD4⁺ T helper cells are required for the activation of effector CTLs via the apoptosis-dependent exogenous pathway for MHC I antigen presentation.

FIGS. 5A-B show that CD8⁺ T cells stimulated via the exogenous MHC I pathway undergo proliferation in the absence of CD4⁺ help.

FIG. 6 depicts that cross-presentation of antigen to CD8⁺ T cells in the absence of CD4⁺ T cell help results in proliferation and subsequent apoptotic cell death.

FIGS. 7A-E shows that DC maturation is required for the cross-tolerization of influenza-specific CD8⁺ T cells.

FIG. 8 shows that CD40L dose-responsively substitutes for CD4+ help.

FIGS. 9A-C shows that FK506, but not cyclosporin A, inhibits cross-priming by affecting the dendritic cell.

FIGS. 10A-C shows that FK506 selectively affects the exogenous MHC I pathway.

FIGS. 11A-D shows that FK506 does not inhibit phagocytosis, dendritic cell maturation nor generation of MHC I/peptide complexes.

FIG. 12 shows that FK506 acts to inhibit cross-priming by blocking signal ing of TNF superfamily members.

FIG. 13 depicts the method for assaying of tolerance versus ignorance.

FIGS. 14A-C shows that treatment of DCs with FK506 results in skewing the cross-presentation of antigen toward the tolerization of antigen-specific CD8+ T cells.

### DETAILED DESCRIPTION OF THE INVENTION

Previously described in-vivo models demonstrated that tissue-restricted antigen may be captured by bone marrow derived cells and cross-presented for tolerization of CD8⁺ T cells. While these studies have shown peripheral deletion of CD8⁺ T cells, the mechanism of antigen transfer and the nature of the antigen presenting cell (APC) remained heretofore undefined. The present inventors, by establishing the first in-vitro system for the study of cross-tolerance, have demonstrated that dendritic cells (DCs) phagocytose apoptotic cells and tolerize CD8⁺ T cells only when CD4⁺ helper cells are absent. Employing this system, it was also found that the same mature DC, which cross-presenting antigen derived from apoptotic cells, is required for both priming and tolerizing. These data indicate the need for both mature DC and the presence of CD4⁺ T cells in cross-priming, and the need for mature DC but the absence of effective CD4 T cells for tolerization. These observations form the basis of the invention and the ex-vivo and in-vivo methods for tolerization described herein.

The new culturing methodology for achieving in-vitro tolerance has been prepared as follows: apoptotic cells are co-culture with immature DCs in the presence or absence of a maturation stimulus, mimicking events that occur in the periphery. The DCs are then harvested after 36-48 hours, and tested for their ability to activate versus tolerize influenza-specific T cell responses, an interaction which likely occurs in the draining lymph organs. Specifically, peripheral blood was obtained from normal donors in heparinized syringes and PBMCs were isolated by sedimentation over Ficoll-Hypaque (Pharmacia Biotech). T cell enriched and T cell depleted fractions were prepared by rosetting with neuraminidase-treated sheep red blood cells. Immature dendritic cells (DCs) were prepared from the T cell depleted fraction by culturing cells in the presence of granulocyte and macrophage colony-stimulating factor (GM-CSF, Immunex) and interleukin 4 (IL-4, R & D Systems) for 7 days. 1000 U/ml of GM-CSF and 500-1000 U/ml of IL4 were added to the cultures on days 0, 2 and 4. To generate mature DCs, the cultures were transferred to fresh wells on day 6-7 and monocyte conditioned media (MCM)(M. L. Albert, B. Sauter, N. Bhardwaj, Nature 392, 86-9, 1998) or a mixture of 50 U/ml tumor necrosis factor-alpha (TNF-α, Endogen) and 0.1 μM prostaglandin E-2 (PGE-2, Sigma Co.) was added for an additional 1-2 days. At day 6-7, >95% of the cells were CD14-, CD83+, HLA-DRlo DCs. Post-maturation, on day 8-9, 70-95% of the cells were of the mature CD14-, CD83+, HLA-DRhi phenotype. CD4+ and CD8+ T cells were further purified to >99% purity by positive selection using the MACS column purification system (Miltenyi Biotech.).

The foregoing system may be used in any number of ways: to identify critical components of a cellular immune response, such as but not limited to enhancing or blocking surface receptors required for the maturation of the dendritic cell; enhancing, blocking, agonizing, antagonizing the interaction between the dendritic cell and T cells through the engagement of TNF superfamily cytokines and their receptors; defining surface receptors capable of delivering antigen to the DCs for purposes of cross-tolerizing CD8⁺ T cells; identifying novel ways to direct antigen for the priming vs. tolerization of CD8=T cells, among others.

As mentioned above, dendritic cells (DCs) phagocytose apoptotic cells, process antigen derived therefrom and activate class I-restricted CD8⁺ T cells [Albert, M. L., Sauter, B. & Bhardwaj, N. Dendritic cells acquire antigen from apoptotic cells and induce class I-restricted CTLs. Nature 392, 86-89 (1998)]. It is demonstrated in the examples herein that the activation of CD8⁺ T cells via this exogenous pathway requires CD4+ helper T cells. This helper cell requirement can be substituted by soluble TRANCE and CD40L, among other factors. As defined herein, “effective CD4+ help” and syntactic variants thereof refer to various means for intervening in the aforesaid participation of CD4+ T cell help, or blocking dendritic cell—CD4+ T cell engagement, thus resulting in immune tolerance to the pre-selected antigen. Effective CD4+ help includes the presence of CD4+ cells, the presence of CD4+-T-cell-derived ligands such as but not limited to TRANCE, CD40L, OX40 ligand and TWEAK that interact with receptors on dendritic cells, and necessary signaling events consequent to CD4+ T-cell engagement. Thus, the absence of effective CD4+ help is defined by any one or more of the following: absence of CD4+ T cells, absence of or blocking the interaction of TRANCE, CD40L, OX40 ligand, TWEAK, or another TNF superfamily member and its receptor; or blocking signal transduction related to CD4+ T-cell engagement.

In addition to the use of the foregoing tolerance in-vitro model system for identifying and evaluating components that have the ability to skew the immune response toward a pre-selected antigen in the direction of tolerance, various therapeutic methods derive therefrom. These are broadly directed to either ex-vivo or in-vivo methods for tolerizing the immune system to a pre-selected antigen. As noted above, these methods take advantage of the discoveries herein that the combination of maturation of the dendritic cell and the participation of CD4 T cell help is required for the cross-priming of the immune response to form effector T cells capable of recognizing the pre-selected antigen that originated from a cell source other than the dendritic cell, and thus the exploitation of these observations in permitting dendritic cell maturation and the absence of effective CD4 T cell help in skewing the immune response towards tolerance. In the practice of the invention, upregulation or surface expression of co-stimulatory molecules characteristic of dendritic cell maturation are triggered or not interfered with, such as but not limited to TNF, PGE2, LPS, monocyte conditioned media, CpG, which are agents capable of inducing dendritic cell maturation. With regard to the elimination of effective CD4+ help, in the methods of the invention, this takes the form of various means for either eliminating the CD4+ T cells themselves; or intervening in the activity of one or more members of interacting, extracellular (secreted or cell surface) CD4+ T cell or dendritic cell products, such as one or more receptors or their ligands; or by means of interfering with the signaling induced by the presence of the cells or the consequence of the interaction of the above-mentioned extracellular products. In practice, such means include but are not limited to eliminating CD4+ T cells from an ex-vivo system or from the in-vivo site of immune activation, or preventing the consequences of interaction between CD4+ T helper cells and dendritic cells by interfering with the interaction between various receptor-ligand pairs known to be able to substitute for CD4+ T cell help in the generation of CD8 effector cells, such as, by way of non-limiting example, CD40 and CD40 ligand, TRANCE and TRANCE receptor, OX40 and OX40 ligand, DR3 and TWEAK, and interfering with other ligand-receptor interactions which abrogate the participation of effective CD4+ help on the development of a cellular immune response (i.e., priming). In addition, the downstream signal transduction pathways consequent to the interaction between the aforementioned receptor-ligand pairs (DC-CD4+ T-cell engagement) are also effective targets for eliminating effective CD4+ help. Such may be achieved, for example, using compounds which antagonize FK binding protein (FKBP), such as FK-506, or compounds that antagonize TOR, such as rapamycin, either of which are also effective at achieving the desired tolerance. These various routes for abrogating dendritic cell maturation or effective CD4+ T cell help may be selected for the particular method undertaken to induce ignorance or tolerance, and one or a combination of such agents may be employed.

Another effective route for the inhibition of DC-CD4+ T-cell engagement is the inhibition of the generation of the MHC II/peptide complex. This may be achieved in the practice of the present invention by the use of agents which inhibit formation of mature forms of MHC II/peptide complexes within the dendritic cell, by way of non-limiting example, preventing cleavage of the invariant MHC II chain using one or more cathepsin inhibitors, blocking loading of peptides by inhibiting HLA-DM, preventing successful antigen degradation and MHC II peptide epitope by inhibiting cathepsin D or alternative proteases, or by inhibiting transport of MHC II/peptide complexes to the cells surface.

Thus, in the practice of ex-vivo methods for inducing tolerance to a pre-selected antigen, dendritic cell maturation is required together with inhibition of effective CD4+ help. In an example of the practice of the invention, tolerance to a pre-selected antigen may be induced either in vivo or ex vivo by providing a pre-selected antigen such that dendritic cells can process the antigen, mature, and present antigen-derived peptides in complexes with MHC I, for presentation to CD8⁺ T cells. Thus, in this aspect of the invention, signals permitting dendritic cell maturation and peptide presentation are necessary. In addition, effective CD4+ T cell help is blocked. For ex-vivo methods, in a non-limiting example,

4. peripheral blood mononuclear cells (PBMC) are isolated from a whole blood sample from a patient scheduled for a renal transplant from an unrelated donor;

5. dendritic cells are isolated from the PBMC;

6. cells from the donor of the kidney are obtained and apoptosis induced therein by exposure to radiation;

7. the dendritic cells and apoptotic cells are admixed in the presence of the dendritic cell maturation stimulatory molecules PGE2 and TNF, and also in the presence of agents which abrogate effective CD4+ help, including a monoclonal antibody to TRANCE and FK-506; alternatively FK506, rapamycin, or the combination may be used, in addition to the aforementioned monoclonal antibody or antibodies;

8. after a period of time, the cellular portion of the mixture or a part thereof is infused into the patient.

The result is the tolerization of antigen-specific CD8+ cells in the patient.

Numerous variations in the foregoing protocol may be employed. The donor antigen may be provided to the dendritic cells by other means than using the donor individual's own cells, such as loading an alternate or different cell type with the donor antigen, and then inducing apoptosis therein. Alternatively, cells may be transfected to express the various antigens towards which tolerance is desired, for feeding to dendritic cells. Antigen may also be bound in ‘artificial’ apoptotic cell/body, lipid bilayers containing anionic phospholipids such as phosphatidylserine, a receptor for engagement with αᵥβ₅ on the DC such as lactadherin or Dell, and other protein and lipid products required to model an ‘artificial’ apoptotic cell/body. The antigen may also be contained within an exosome or be part of an antigen/antibody immune comples. In another example, artificial antigen presenting cells may be used in place of the recipient individual's PBMC as a source. The means by which the antigen is exposed to the dendritic cells is not limited and the foregoing examples merely exemplary of several among many ways to carry out this step of the method of the invention.

Various other dendritic cell maturation stimuli as well as inhibitors of effective CD4+ T cell help may be used, as described throughout herein. Stimulators such as TNF-alpha, PGE2, lipopolysaccharide, and CpG-DNA are merely exemplary.

Prior to reinfusion of the ex-vivo mixture, purification of the ex-vivo cells from the mixture of added reagents is optional, depending on the level of agents added to and retained activity present with the cells. Cells may be washed by any means prior to infusion.

As mentioned above, the ex-vivo system eliminates effective CD4+ help by a means such as:

i) eliminating CD4+ cells from the ex-vivo system;

ii) including CD4+ cells in the ex-vivo system, but including at least one inhibitor of the interaction between a TNF superfamily member and its receptor;

iii) including CD4+ cells in the ex-vivo system, but including an inhibitor of signal transduction from the foregoing steps; and/or

iv) inhibiting generation of MHC II/peptide complexes on the dendritic cells or preventing MHC II/peptide complex engagement with the CD4+ T cell receptor.

In particular, examples (ii)-(iv) above are preferred as they will also prevent engagement of the DC and CD4⁺ T helper cell after DC infusion. These methods achieve the desired abrogation or diminution of effective CD4+ T cell help. Various combinations of the four foregoing methods may be employed in combination, depending on the purity of the cellular population, or other considerations such as the effectiveness of inhibiting a single receptor-ligand or signal transduction pathway member. Such determination and resulting selection of agents and/or methods for inhibiting effective CD4+ T cell help will be readily determinable by one of skill in the art. Preferably, dendritic cells are treated with the aforementioned inhibitors prior to reinfusion into the individual where CD4⁺ T cells exist. The agent is applied such that the DC receptors are prevented from engaging with antigen-specific CD4+T cells; the signaling of the DC TNF superfamily receptors are blocked; or the generation of the MHC I/peptide complex is inhibited so that by one or a plurality of absent routes, the DC can not engage the CD4+T cell.

Examples of such reagents include but are not limited to blocking antibodies, receptor decoys, small molecule inhibitors, membrane permeable drugs which inhibit signal transduction downstream from one of the foregoing steps. The latter may be achieved by, for example, interfering with FKBP activity or with TOR activity. These may be achieved by the use in the exvivo system by using FK-506, or rapamycin, respectively. They also may be used systemically in the practice of the in-vivo methods of the invention, for example, when dendritic cells are attracted locally or antigen is supplied to dendritic cells locally. These are merely examples of agents with the desired activity which may be used effectively to achieve the desired tolerance of the immune system to the pre-selected antigen.

Following the above steps, the cellular components of the ex-vivo system may be introduced into the patient. As will be seen below, cells treated as above result in the skewing of the immune response towards the tolerization of antigen-specific CD8+ cells.

In the practice of the invention in vivo, temporary localization of the cellular components is desirable. For example, dendritic cells may be attracted to a particular site, such as a subdermal site, in the body by placement on the skin of a transcutaneous delivery device comprising a dendritic cell chemoattractant such as but not limited to ligands for CCR6 such as 6-C-kine. The delivery device also delivers a pre-selected antigen, as well as a blocker of effective CD4+ help, such as an FKBP or TOR antagonist. Examples include but are not limited to topical FK-506 and rapamycin. Antigen processing by the dendritic cell may also be inhibited by the local inclusion of an agent which inhibits the generation of MHC II/peptide complexes on the dendritic cell, by, for example, preventing cleavage of the invariant chain using cathepsin inhibitors, blocking loading of peptides by inhibiting HLA-DM, preventing successful antigen degradation and MHC II peptide epitope by inhibiting cathepsin D or alternative proteases, or by inhibiting transport of MHC II/peptide complexes to the cells surface. Dendritic cells having encountered antigen at the subdermal site, in the absence of effective CD4+ help, or any of the foregoing, will proceed to induce apoptosis of antigen-specific CD8+ T cells, resulting in immune tolerance to the antigen.

The foregoing description of the in-vivo protocol may be modified for various purposes and still be encompassed within the teachings herein. For example, in a condition in which a lesion is present in the body comprising an antigen for which abrogation of an immune response is desired, dendritic cells may be attracted to a lesion using the methods herein, by providing locally at the lesion site a dendritic cell attractant and one or more agents as described above, such as FK-506, to skew the immune response toward tolerance to the antigen present in the lesion. The agent may be given systemically when the attraction of dendritic cells, the provision of the antigen, or both, is locally. In another embodiment, dendritic cells may be trafficked to a site in the body using a chemoattractant as described above, and at the site the antigen being provided to the attracted dendritic cells. The agent to skew the immune response to tolerizing also may be provided locally at the site, or it may be provided systemically. These methods may be carried out for any of the purposes described herein, such as but not limited to preventing or prophylaxing an autoimmune disease, acceptance of transplanted cells, tissues or organs, and abrogating an immune response where an overactive immune response is occurring.

Thus, in an example of an in-vivo protocol, a patch is placed on a psoriatic lesion on the skin of an individual suffering from psoriasis, with the objective of reducing or eliminating autoreactive T cells to the psoriatic antigen. The patch includes a dendritic cell chemoattractant compound (e.g., ligands for CCR6 such as 6-C-kine) and FK-506. After one week, the patch is removed. While not being bound by theory, the patch attracts dendritic cells to the site where they encounter psoriatic antigens in the presence of an agent (local or systemically administered) which blocks effective CD4+ T cell help. The dendritic cells migrate to the lymph nodes where they induce apoptosis in psoriasis-antigen-specific memory CD8+ T cells. Reduced psoriatic pathology is achieved. The present invention may be better understood by reference to the following non-limiting Examples, which are provided as exemplary of the invention. They should in no way be construed, however, as limiting the broad scope of the invention. The examples demonstrate the requirement for dendritic cell maturation and effective CD4+T cell help in inducing crosspriming, and the finding that in the presence of dendritic cell maturation, inhibition of effective CD4 T cell help results in tolerance to the antigen.

### EXAMPLE 1

### Demonstration of the Requirement for Absence of CD4+ T-cell Help in Tolerance

Media.

RPMI 1640 supplemented with 20 μg/ml of gentamicin (Gibco BRL), 10 mM HEPES (Cellgro) and either 1% human plasma, 5% pooled human serum (c-six diagnostics) or 5% single donor human serum was used for DC preparation, cell isolation and culture conditions.

Detection of Antigen-specific T Cells.

ELISPOT assay for IFN-γ release-Immature DCs, apoptotic cells and monocyte conditioned media were incubated together for 2 days to allow antigen processing and DC maturation to occur. The DCs were collected, counted and added to purified T cell populations in plates precoated with 10 μg/ml of a primary anti-IFN-γ mAb (Mabtech). In all experiments, 6.67×10³ DCs were added to 2×10⁵ T cells to give a 1:30 DC:T cell ratio. The cultures were incubated in the plates for 20 hours, at 37° C. and then the cells were washed out. Wells were then incubated with 1 μg/ml biotin-conjugated anti-IFN-γ antibody (Mabtech). Wells were next stained using the Vectastain Elite kit as per manufacturers instructions (Vector Laboratories). Colored spots represented the IFN-γ releasing cells and are reported as spot forming cells/10⁶. Triplicate wells were averaged and means reported.

⁵¹Chromium Release Assay.

Influenza infected monocytes or HeLa cells were triggered to undergo apoptosis (see above), and put in co-culture with DCs and T cells prepared from HLA-A2.1⁺ blood donors. Alternatively, apoptotic cells were co-cultured with immature DCs in the presence of a maturation stimulus for 8-36 hours prior to the establishment of DC-T cell cultures. In CTL assays, responding T cells were assayed after 7 days for cytolytic activity using T2 cells pulsed for 1 hr with 1 μM of the immunodominant influenza matrix peptide, GILGFVFTL (Gotch, F., Rothbard, J., Howland, K., Townsend, A. & McMichael, A. Cytotoxic T lymphocytes recognize a fragment of influenza virus matrix protein in association with HLA-A2. Nature 326, 881-882, 1987; Gotch, F., McMichael, A., Smith, G. & Moss, B. Identification of viral molecules recognized by influenza-specific human cytotoxic T lymphocytes. J Exp Med 165, 408-416, 1987). Specific lysis indicates that the APC had cross-presented antigenic material derived from the apoptotic cell, leading to the formation of specific peptide-MHC class I complexes on its surface. Specific Lysis=(% killing of T2 cells+peptide)−(% killing of T2 cells alone). Background lysis ranged from 0-13%. Influenza-infected DCs served as controls in all experiments and allowed for to determination of the donor's CTL responsiveness to influenza. Other methods used herein may be found described in the other examples below.

Dendritic cells acquire antigen from cells and induce class I-restricted influenza-specific CTLs in a CD4-dependent manner. With a better understanding of the physiologically relevant steps involved in the capture and presentation of antigen derived from apoptotic cells [Albert, M. L. et al. Immature dendritic cells phagocytose apoptotic cells via αᵥβ₅ and CD36, and cross-present antigens to cytotoxic T lymphocytes. J Exp Med 188, 1359-1368 (1998); Sauter, B. et al Consequences of Cell Death. Exposure to necrotic tumor cells, but not primary tissue cells or apoptotic cells, induces the maturation of immunostimulatory dendritic cells. J Exp Med 191, 423-434 (2000)], the culturing methodology was refined as follows: i) apoptotic cells expressing influenza antigen are co-cultured with immature DCs in the presence of a maturation stimulus; ii) DCs are harvested after 36-48 hours and tested for their ability to activate influenza-specific T cell responses. Note, at the time of harvesting, the DCs demonstrate a mature phenotype based on CD83 and HLA-DR^{hi} surface expression. The murine lymphoma cell line EL4 (ATTC #TIB-39) was used as a source of apoptotic cells as they can be efficiently infected with influenza virus, and do not induce significant background T cell activation to murine antigens.

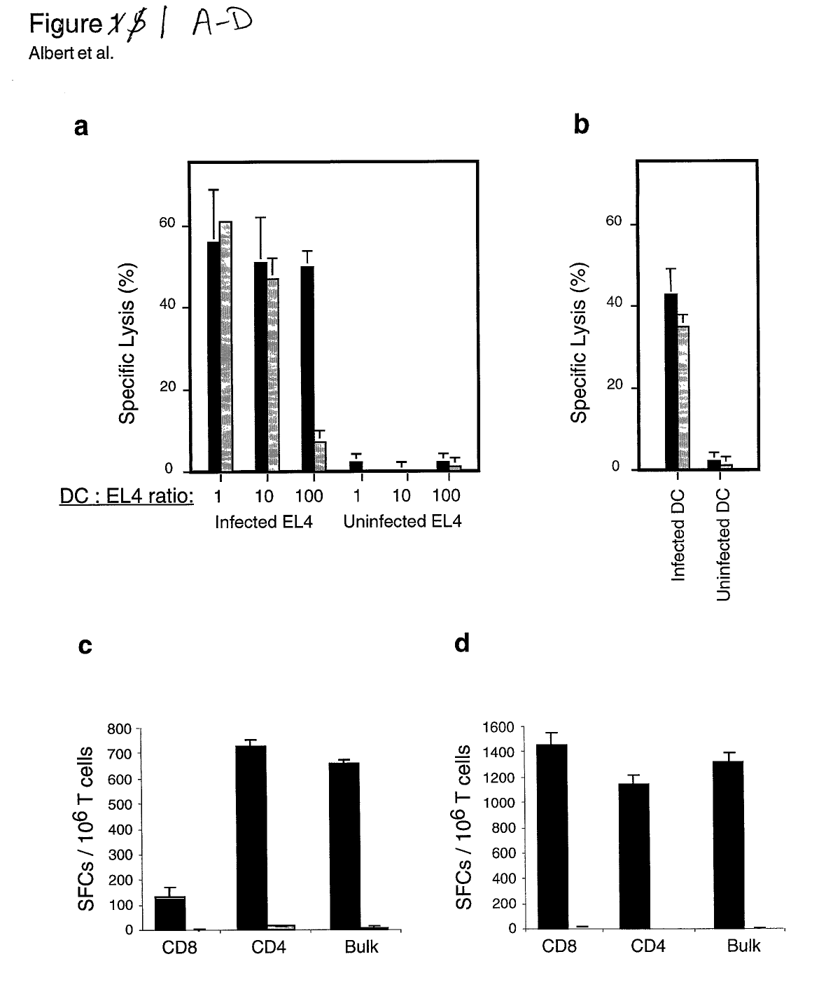

EL4 cells were first infected with influenza A (stain PR/8), and cultured for 6 hours to permit expression of viral proteins. These cells were then irradiated with 240 mJ/sec² of UVB irradiation, to trigger apoptotic cell death. After 8-10 hours, DCs from a HLA-A2.1⁺ donor were co-cultured with the dying EL4 cells. After 48 hours, the DCs were harvested and plated with syngeneic T cells. As shown in FIG. 1, DCs were collected and plated with bulk T cells at a ratio of 1:30 (black bars) or 1:100 (gray bars). After 7 days, responding T cells were tested in a standard ⁵¹Cr assay using T2 cells (a Tap^{-/-}, HLA-A2.1⁺ cell line) pulsed with the immunodominant influenza matrix peptide as targets. Effector: target ratios=25: 1. (FIG. 1A). As a control for the individual's responsiveness to influenza, infected DCs were used to measure the activation of CTLs via the endogenous pathway for MHC I (FIG. 1B). Various doses of influenza infected EL4 cells were co-cultured with DCs for 24-36 hours. The DCs were then collected, counted and plated with either highly purified CD8⁺ T cells, CD4⁺ T cells or mixtures of both (bulk T cells =2:1 CD4:CD8 cells). 6.6×10³ DCs were plated with a total of 2×10⁵ T cells to give a ratio of 1:30. Cells were co-cultured in plates precoated with 10 μg/ml of a primary anti-IFN-γ mAb. After 30-40 hours, the cells were removed and the plates developed as described in methods. Spot forming cells (SFCs) per 10⁶ T cells are reported. Note, uninfected EL4 cells were used as a control, and <2 SFCs/10⁶ T cells were detected (FIG. 1C). Influenza infected and uninfected DCs served as a control. Additionally, the infected DCs allowed for the comparison between the requirement for help in exogenous (FIG. 1C) vs. endogenous (FIG. 1D) MHC I antigen presentation. Results in FIG. 1 are representative of more than 15 experiments and values shown are means of triplicate wells. Error bars indicate standard deviation.

As noted above, influenza-specific CTLs were measured after 7 days in a chromium release assay using T2 cells pulsed with the immunodominant HLA-A2.1 -restricted influenza matrix peptide [Gotch, F., Rothbard, J., Howland, K., Townsend, A. & McMichael, A. Cytotoxic T lymphocytes recognize a fragment of influenza virus matrix protein in association with HLA-A2. Nature 326, 881-882 (1987)]. Influenza specific CTLs were generated in these co-cultures, but not in cultures in which uninfected apoptotic EL4 cells were used (FIG. 1A), nor when DCs were excluded. Influenza infected DCs, presenting antigen via the classical MHC I antigen presentation pathway served as a positive control, and established the individual's prior exposure to influenza (FIG. 1B). This experiment illustrates the two-step process of antigen presentation where the apoptotic cell is captured by the immature DC and only upon maturation may it activate memory CD8⁺ T cells to become effector CTLs. By using this refined culturing method, only 1 apoptotic cell is required per 100 DCs to generate a CTL response as potent as that measured with influenza infected DCs.

The ELISPOT assay, which enumerates the number of T cells producing IFN-γ in response to antigen can also be utilized to measure T cell responses to antigens cross-presented from apoptotic cells. DCs exposed to influenza infected, apoptotic EL4 cells (as described above), were co-cultured with purified CD8⁺ T cells, CD4⁺ T cells or reconstituted bulk T cells (2:1 ratio of CD4:CD8 T cells). After 36-40 hours, the number of IFN-γ producing cells was quantified as described in the methods section. In a representative experiment, 650 SFCs per 10⁶ bulk T cells were detected. To our surprise, when T cell subsets were tested, <130 spot forming cells/10⁶ (SFCs) were detected when purified CD8⁺ T cells were used as the responder cells. When purified CD4⁺ T cells were the responders, 725 SFCs per 10⁶ CD4⁺ T cells were detected (FIG. 1C). As a negative control, uninfected EL4 cells were used as a source of apoptotic cells, and <2 SFCs/10⁶ cells were detected in all groups tested. Again, influenza infected DCs were used as a positive control, and >1450 SFCs per 10⁶ CD8⁺ T cells were measured (FIG. 1D). While this experiment established that CD8⁺ T cells are capable of generating detectable quantities of IFN-γ, it is remained unclear whether the CD4 or the CD8⁺ T cells were producing the IFN-γ in the bulk cultures. Thus, mechanisms of substituting for CD4 helper T cells were evaluated to demonstrate that one could elicit IFN-γ from CD8⁺ T cells via the apoptosis-dependant exogenous pathway.

The next study demonstrated that TRANCE Receptor and CD40 receptor activation substitute for CD4⁺ helper T cells in supporting the cross-priming of CD8⁺ T cells. Recent reports have suggested that ligation of the TNF receptor family member, CD40, on DCs replaces the requirement for CD4+help in in-vivo cross-presentation models [Bennett, S. R. et al. Help for cytotoxic-T-cell responses is mediated by CD40 signalling. Nature 393, 478-480 (1998); Schoenberger, S. P., Toes, R. E., van der Voort, E. I., Offringa, R. & Melief, C. J. T-cell help for cytotoxic T lymphocytes is mediated by CD40-CD40L interactions. Nature 393, 480-483 (1998); Lanzavecchia, A. Immunology. License to kill. Nature 393, 413-414 (1998); Ridge, J. P., Di Rosa, F. & Matzinger, P. A conditioned dendritic cell can be a temporal bridge between a CD4⁺ T-helper and a T-killer cell. Nature 393, 474-478 (1998)]. Whether CD40 activation might replace CD4 help in the cross-priming of CD8 effector cells by DCs which have captured apoptotic cells was tested. Additionally, a potential role for TRANCE (TNF-related activation-induced cytokine) was evaluated, as it shares several of the functional properties of CD40L [Bachmann, M. F. et al. TRANCE, a tumor necrosis factor family member critical for CD40 ligand-independent T helper cell activation. J Exp Med 189, 1025-1031 (1999)].

Immature DCs were co-cultured with influenza-infected apoptotic EL4 cells and induced to undergo maturation. After 36 hours, the DCs were added to purified CD8⁺ T cells. In addition, either hCD8-TRANCE [generation of reagent described in Wong, B. R. et al. TRANCE (tumor necrosis factor [TNF]-related activation-induced cytokine), a new TNF family member predominantly expressed in T cells, is a dendritic cell-specific survival factor. J Exp Med 186, 2075-2080 (1997)] or mCD8-CD40L was added to the co-cultures. After 40 hrs, the number of SFCs was enumerated by standard ELISPOT assays.

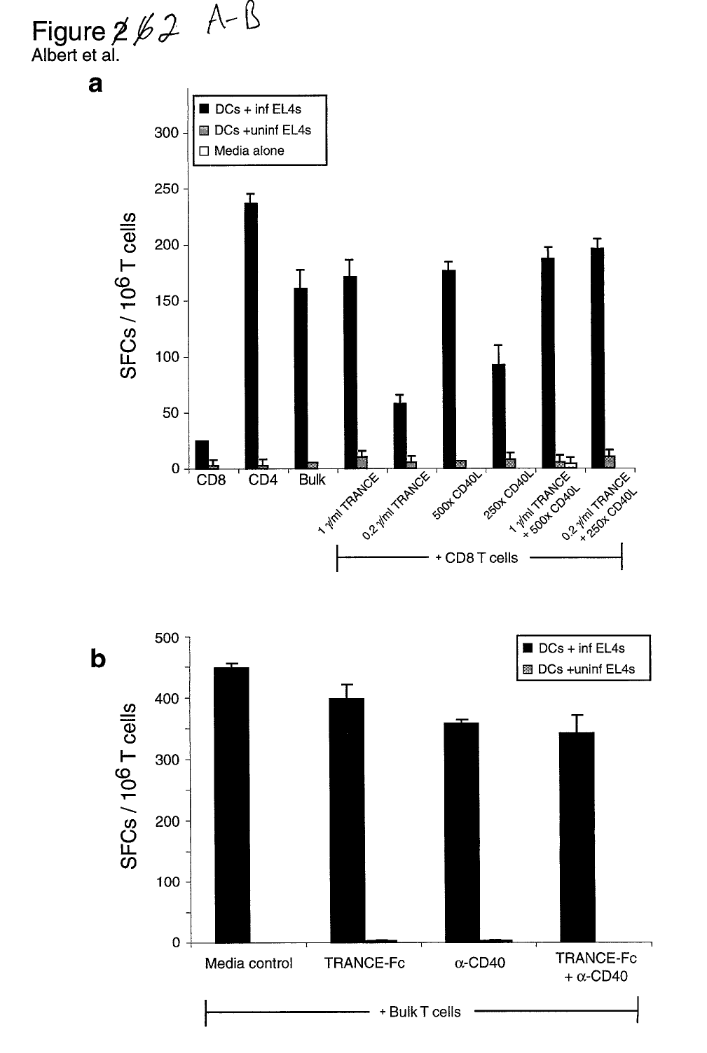

Co-cultures were established as in FIGS. 1C and D. Either hCD8-TRANCE, mCD8-CD40L or both were added to wells containing purified CD8⁺ T cells at the initiation of the DC-T cell co-culture period. IFN-γ producing cells were quantified by ELISPOT assay and SFC/10⁶ cells are reported (a). Reconstituted cultures of bulk T cell (2:1 CD4:CD8 cells) were incubated with DCs charged with apoptotic cell antigen, in the presence of reagents capable of inhibiting the TRANCE/TRANCE-receptor interaction (soluble TRANCE-Fc), and/or the CD40L/CD40 receptor pair (α:-CD40). These reagents were added at a concentration of 10 μug/ml (b). Experiments in FIG. 2 are representative of greater than 10 experiments and values shown are means of triplicate wells. Error bars indicate standard deviation.

Five-10 times the number of IFN-γ producing CD8⁺ T cells could be detected in wells that had received either TRANCE or CD40L, as compared to media alone (FIG. 2A). These pathways are apparently additive, as sub-optimal concentrations of TRANCE and CD40L facilitated efficient cross-priming of antigen-specific T cells when placed in co-culture together. While sufficient to substitute for CD4 help, other pathways are likely to participate as it was not possible to inhibit CD4 cells from providing cognate help using soluble TRANCE receptor fusion protein (TR-Fc, described in Fuller, K., Wong, B., Fox, S., Choi, Y. & Chambers, T.J. TRANCE is necessary and sufficient for osteoblast-mediated activation of bone resorption in osteoclasts. J Exp Med 188, 997-1001, 1998) in combination with a blocking monoclonal antibody against the CD40 receptor (FIG. 2B). This was confirmed by chromium release assay.

Several possibilities might account for the ability of TRANCE receptor and CD40 ligation to induce the cross-priming of CD8⁺ T cells. One explanation might be the ability of TRANCE and CD40L to induce DC maturation [Cella, M. et al Ligation of CD40 on dendritic cells triggers production of high levels of interleukin-12 and enhances T cell stimulatory capacity: T-T help via APC activation. J Exp Med 184, 747-752 (1996)]. As the DC population is mature when placed into co-culture with the T cells (as defined by surface expression of CD83 and high levels of HLA-DR), alternate interpretations appear to account for the results and provide the surprising and unexpected results on which the invention herein is based. The activation of TRANCE and CD40 receptors results in increased DC survival [Wong, B. R. et al. TRANCE (tumor necrosis factor [TNF] -related activation-induced cytokine), a new TNF family member predominantly expressed in T cells, is a dendritic cell-specific survival factor. J Exp Med 186, 2075-2080 (1997)]. Accordingly, more DCs would be available to activate T cells. However, no significant difference in viability was noted between TRANCE and CD40L-treated vs. untreated groups during the 40 hr time course used in the ELISPOT assays.

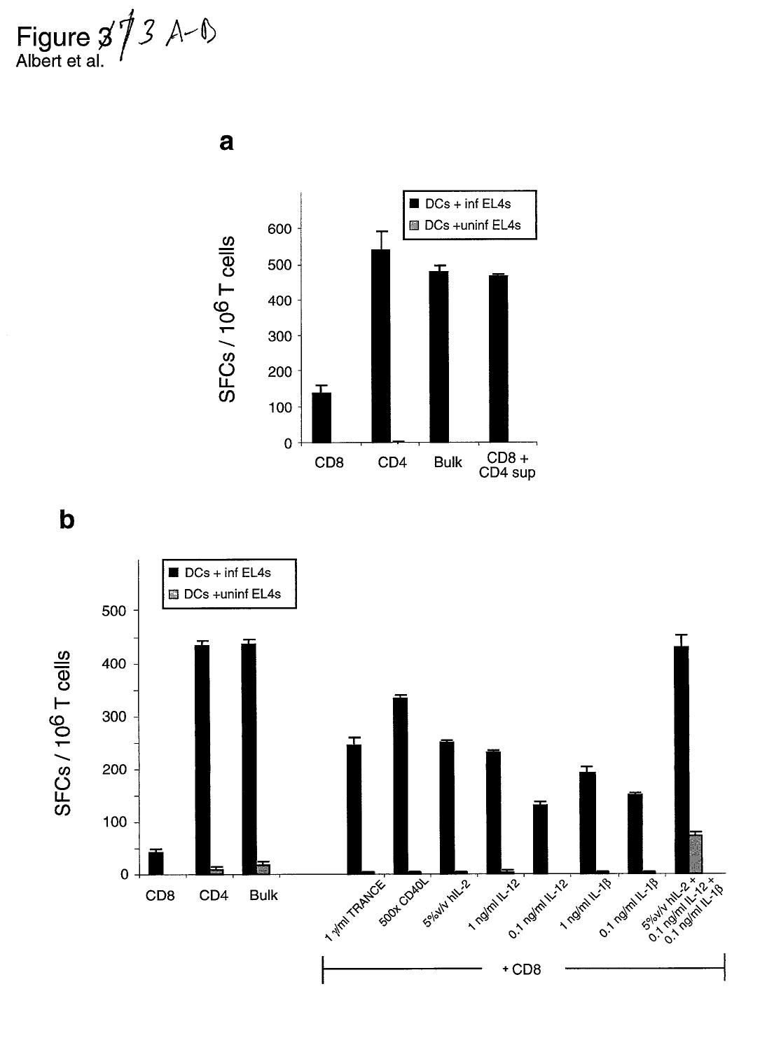

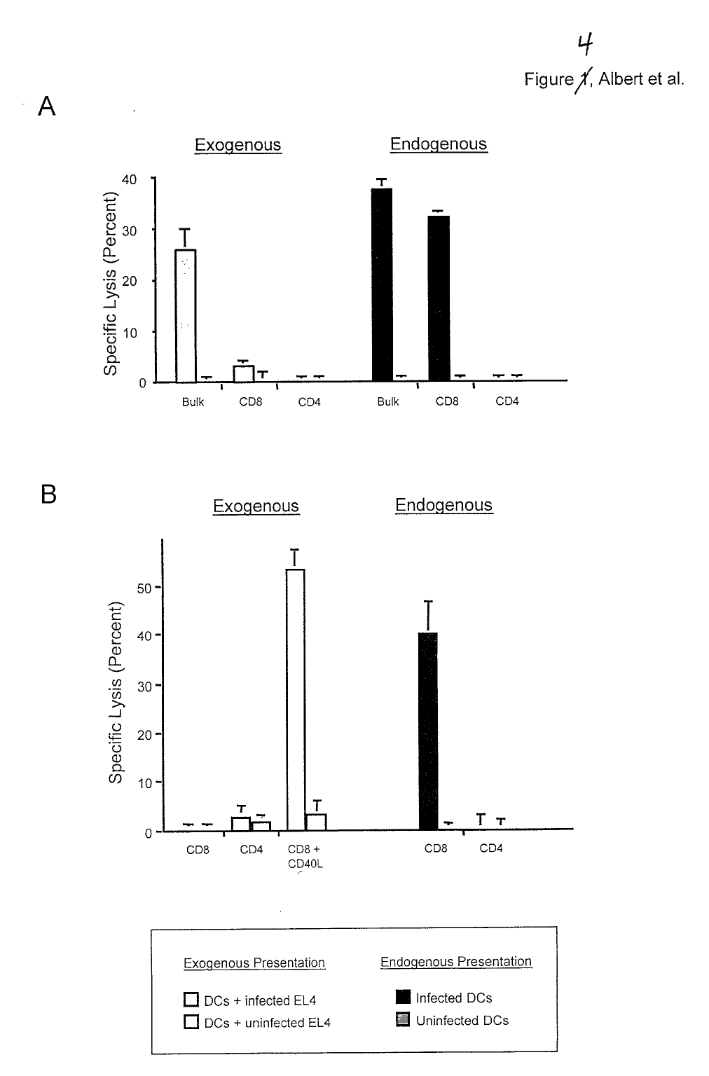

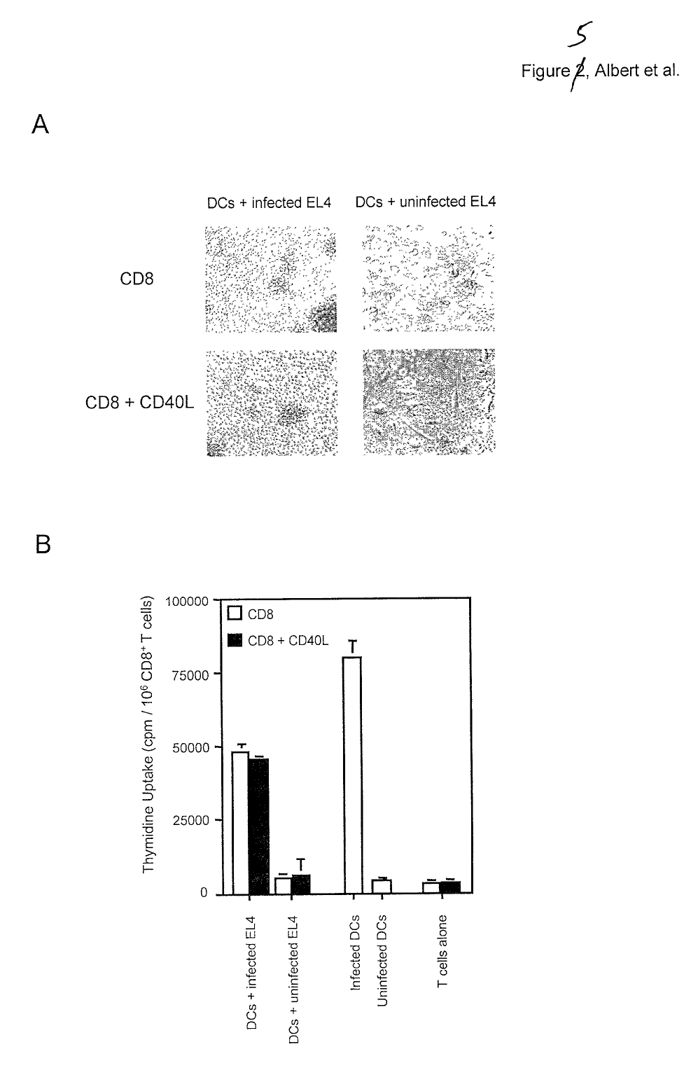

TRANCE receptor and CD40 activation also results in the increased production of several cytokines (e.g. IL-6, TNF-α, IL-15). Whether cognate help (provided by CD4 helper cells or soluble CD40L and TRANCE) could be substituted by supernatants isolated from cultures containing purified CD4⁺ T cells and DCs which had cross-presented influenza infected, apoptotic EL4 cells, was also tested. Co-cultures were established as described above. Supernatants were harvested from wells containing CD4⁺ T cells and DCs which had cross-presented influenza infected EL4 cells. These supernatants were added to wells containing purified CD8⁺ T cells and DCs which had cross-presented influenza infected EL4 cells. IFN-γ producing cells were evaluated as described above. (a). Titrated doses of rhIL-12, rhIL-1β as well as purified hIL-2 were added to wells containing purified CD8⁺ T cells and DCs which had cross-presented influenza infected EL4 cells. ELISPOT assays were performed and SPC/10⁶ cells are reported (b). Experiments in FIG. 3 are representative of 5 experiments and values shown are means of triplicate wells. Error bars indicate standard deviation.

As shown, this supernatant also allowed for the activation of influenza-specific CD8⁺ T cells (FIG. 3A). Titrated doses of rhIL-12, rhIL-1β as well as purified hIL-2 were added to wells containing purified CD8⁺ T cells and DCs which had cross-presented influenza infected EL4 cells. ELISPOT assays were performed and SPC/10⁶ cells are reported.

To identify the cytokines with this activity, the inventors attempted to detect IL-2, IL-12 and TNF-α by ELISA in these supernatants derived from the CD4⁺ T cells/DC cultures described above. In each case, cytokine levels were below the limit of detection. Therefore, whether exogenous recombinant cytokines might substitute for the lack of CD4⁺ T cell help was directly tested. Addition of IL-2, IL-1β or IL-12 all supported the release of IFN-γ by influenza-specific CD8⁺ T cells (FIG. 3B). In combination, these cytokines worked additively to maximally activate the antigen-specific T cells as evident by the increased number of IFN-γ producing cells (FIG. 3B). As the concentrations of IL-2, IL-1β and IL-12 required is non-physiologic, it is likely that TRANCE receptor and CD40 ligation act via additional mechanisms to ‘license’ DCs to cross-prime CD8⁺ T cells. Taken together, this data suggests the following model: immature DCs capture apoptotic cells, and in the presence of a maturation stimulus and cognate CD4 T cell help, the DC is capable of activating antigen-specific CD8⁺ T cells. The cognate interaction between the DC and the CD4 T cell includes but is not limited to TRANCE-TRANCE-R or CD40L-CD40.

### EXAMPLE 2

### The Role of Dendritic Cell Maturation in Cross-tolerance

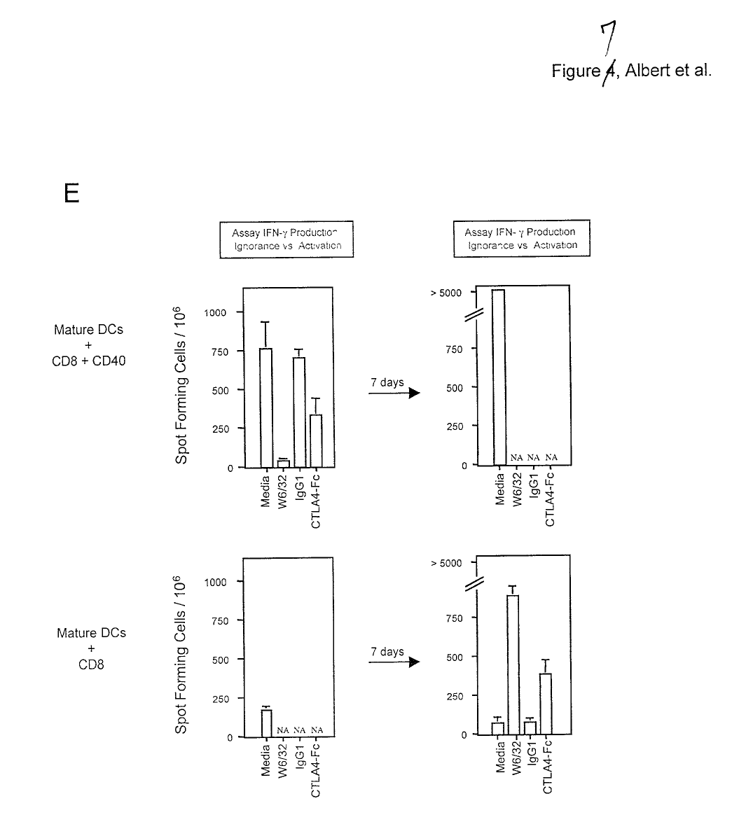

In these experiments, the murine lymphoma cell line, EL4, was used as a source of apoptotic material. The mouse lymphoma cell line EL4 (ATTC #TIB-39) was used as a source of apoptotic cells as they can be efficiently infected with influenza virus, and do not induce tjO significant background T cell activation to mouse antigens (see FIG. 4 and FIG. 7). The EL4 cells were infected with influenza and apoptosis was triggered using a 60UVB lamp (Derma Control Inc.), calibrated to provide 2 mJ/cm²/sec. These cells undergo early apoptotic death within 8-10 hours. Cell death was confirmed using the Early Apoptosis Detection Kit (Kayima Biomedical). To ensure that the uptake of early apoptotic cells was being studied, the kinetics of death were carefully worked out. Six-10 hours post-irradiating, EL4 cells first externalize PS on the outer leaflet of their cell membrane, as detected with Annexin V. By 10-16 hours, these cells were TUNEL positive. It was not until 36-48 hours later that the majority of cells included trypan blue into the cytoplasm, an indicator of secondary necrosis.

Cells were infected with influenza A (strain PR/8), and cultured for 5-6 hours to permit expression of viral proteins. These cells were then induced to undergo apoptosis and co-cultured with immature DCs in the presence of a maturation stimulus. DCs were harvested after 36-48 hrs, and plated with syngeneic T cells (see above). To test for the generation of influenza-specific effector CTLs, cytotoxicity assays were performed using influenza matrix peptide pulsed targets cells (M. L. Albert, B. Sauter, N. Bhardwaj, Nature 392, 86-9, 1998).

As previously reported, DCs are capable of processing exogenous antigen derived from apoptotic cells for the activation of influenza specific CTLs from bulk T cell populations. FIG. 4A shows EL4 cells were infected with influenza and incubated for 5-6 hrs to permit expression of viral proteins. The cells were then irradiated with 240 mJ/sec² of UVB, triggering apoptotic cell death. After 8-10 hrs, 10⁶ immature HLA-A2.1⁺ DCs were co-cultured with 5×10⁶ apoptotic EL4 cells in the presence of a maturation stimulus. DCs were harvested at 36-48 hrs and 6.67×10³ DCs were co-cultured with 2×10⁵ highly purified syngeneic CD8⁺ T cells, CD4⁺ T cells or reconstituted bulk T cells (CD8⁺/CD4⁺ ratio=1:2). Directly infected DCs, presenting antigen via the ‘classical’ endogenous MHC I presentation pathway served as a positive control for the generation of influenza-specific CTLs. After 7 days, cytolytic activity was tested using T2 cells (a TAP0/0, HLA-A2.1⁺ cell line) pulsed with the immunodominant influenza matrix peptide. Specific lysis was determined by subtracting the percent killing of the control targets, unpulsed T2 cells. Effector: target ratio=25:1. In FIG. 4B, DCs were charged with antigen as described above, and co-cultured with syngeneic CD8⁺, CD4⁺ or CD8⁺+CD40L. After 7 days, cytolytic activity was tested as described. In all experiments (FIGS. 4A, 4B), uninfected EL4 cells and uninfected DCs served as the negative controls for presentation of antigen via the exogenous vs. endogenous pathways, respectively. Values are means of triplicate wells and error bars indicate standard deviation. Results in FIG. 4 are representative of >10 experiments.

Influenza infected DCs, presenting antigen via the ‘classical’ endogenous MHC I antigen presentation pathway, served as a positive control (FIG. 4A). Unexpectedly, when purified CD8⁺ T cells were tested, it was not possible to elicit influenza-specific effector CTLs via the exogenous pathway. In contrast, directly infected DCs activated purified CD8⁺ T cells in the absence of CD4⁺ T cells (FIG. 4A) (N. Bhardwaj et al., J Clin Invest 94, 797-807, 1994). As expected, no cytolytic response was detected when using purified CD4⁺ T cells (FIG. 4A). These results illustrated distinction regulatory mechanisms controlling the ability of the exogenous vs. endogenous pathway to stimulate CD8⁺ T cells.

To better define this requirement for CD4⁺ T cell help in the exogenous pathway for MHC I antigen presentation, strategies were evaluated for substituting for the CD4⁺ T cells. Recent reports have suggested that the role of CD4+T cell/DC engagement is to provide CD40 stimulation to the DC [S. R. Bennett et al., Nature 393, 478-80 (1998); S. P. Schoenberger, R. E. Toes, E. I. van der Voort, R. Offringa, C. J. Melief, Nature 393, 480-3 (1998); J. P. Ridge, F. Di Rosa, P. Matzinger, Nature 393, 474-8 (1998); Z. Lu et al., J Exp Med 191, 541-50 (2000)]. Whether CD40 activation might replace CD4⁺ help was therefore tested, permitting the activation of CD8⁺ T cells via the exogenous pathway. Immature DCs were co-cultured with influenza-infected apoptotic EL4 cells and induced to undergo maturation. After 36-48 hours, the DCs were added to purified CD8⁺ T cells in the presence of CD40L (Alexis Biochemical) or agonistic CD40 mAb (Mabtech, clone S2C6). Cultures in which apoptotic cell-loaded DCs had been treated with a stimulus for CD40 were now capable of activating the purified CD8⁺ T cells, indicating that CD40 activation could bypass the requirement for CD4⁺ T cell help (FIG. 4B). While sufficient to substitute for CD4⁺ help, other pathways are also likely to participate as it was not possible to inhibit CD4⁺ cells from providing cognate help using blocking CD40 antibodies. The findings in FIG. 4 were confirmed by ELISPOT assay and FIG. 4C), demonstrating a helper cell requirement for the production of IFN-gamma and the generation of effector CTLs via the exogenous pathway.

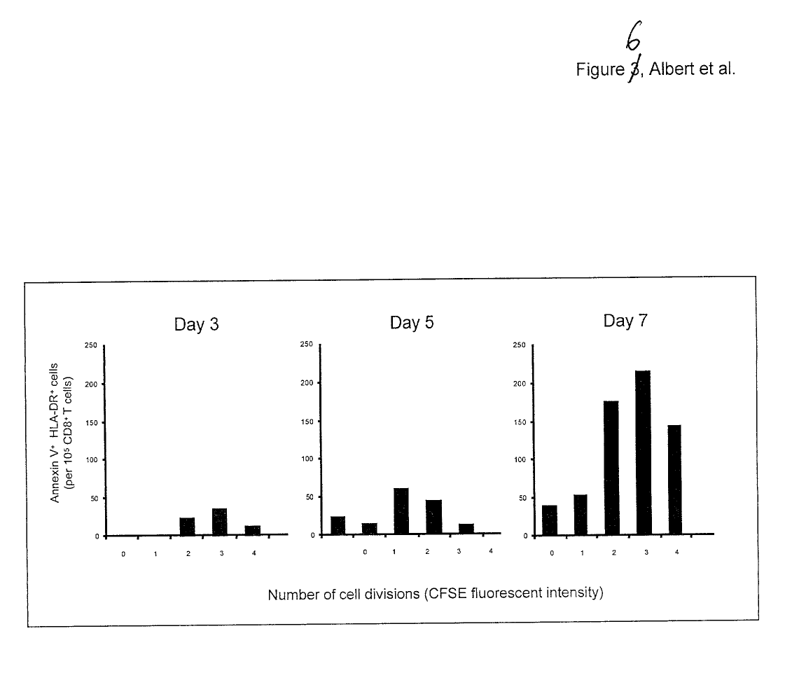

While CD8+T cells did not become effector CTLs in response to DCs cross-presenting influenza infected apoptotic cells (FIG. 5), evidence for antigen-dependent proliferation during the 7 days of culture was detected. In FIG. 5A, immature dendritic cells were co-cultured with influenza infected apoptotic EL4 cells in the presence of a maturation stimulus. After 36-48 hours, DCs were harvested and cultured with syngeneic CD8⁺ T cells in the presence or absence of 1.0 ug/ml CD40L. After 5 days the cultures were imaged by phase contrast using a 20× objective on a Zeiss Axiovert. In FIG. 5B, these cultures were then incubated in the presence of 4 μCi ³H-thymidine for 16 hours T cells and cells were harvested onto a glass fiber filter (EG&G Wallac) and analyzed on a Microbeta Triblux liquid scintillation counter (EG&G Wallac). Note, influenza-infected DCs served as positive control as described in FIG. 4B. T cells alone serve as a control for background levels of thymidine incorporation. Uptake is reported as counts per minute per 10⁶ CD8⁺ T cells; values are means of triplicate wells and error bars indicate standard deviation. Data in FIG. 5 is representative of >5 experiments.

This proliferative response was quantified by ³H-Thymidine incorporation. Influenza infected or uninfected apoptotic cells were co-cultured with 2×10⁵ purified T cells and DCs. Co-cultures were established as described above. After 4.5 days, assays were pulsed with 4 μCi/ml ³H-thymidine and harvested 16 hours later. Indeed, the cellular proliferation detected in co-cultures containing purified CD8⁺ versus those exposed to DCs in presence of CD40L were found to be equivalent (FIG. 5B). One possibility is that the proliferating cells were being deleted, thus accounting for the in vivo phenomenon of cross-tolerance (C. Kurts et al., J Exp Med 186, 2057-62, 1997). To directly test this possibility, an assay was established to detect T cell apoptosis while tracking the number of cell divisions. T cells were labeled with the fluorescent dye CFSE at 0.1 μM and co-cultured for 7 days with DCs as described above. CFSE-labeled cells divide and daughter cells receive approximately half the fluorescent dye, thus allowing for the monitoring of proliferation through 4-5 rounds of cell division. In studying natural immune responses in humans, one is limited by low precursor frequencies of antigen-specific cells (0.02-1.2% influenza specific precursors, range determined in screen of >100 blood donors, as compared to studies that employ TCR-transgenic mice. Thus, to assess cell death in the antigen-responsive cells, T cell populations were labeled with an HLA-DR⁺ mAb. HLA-DR expression showed the lowest background labeling in unstimulated T cells as compared to other activation markers such as CD25, CD38 and CD69.

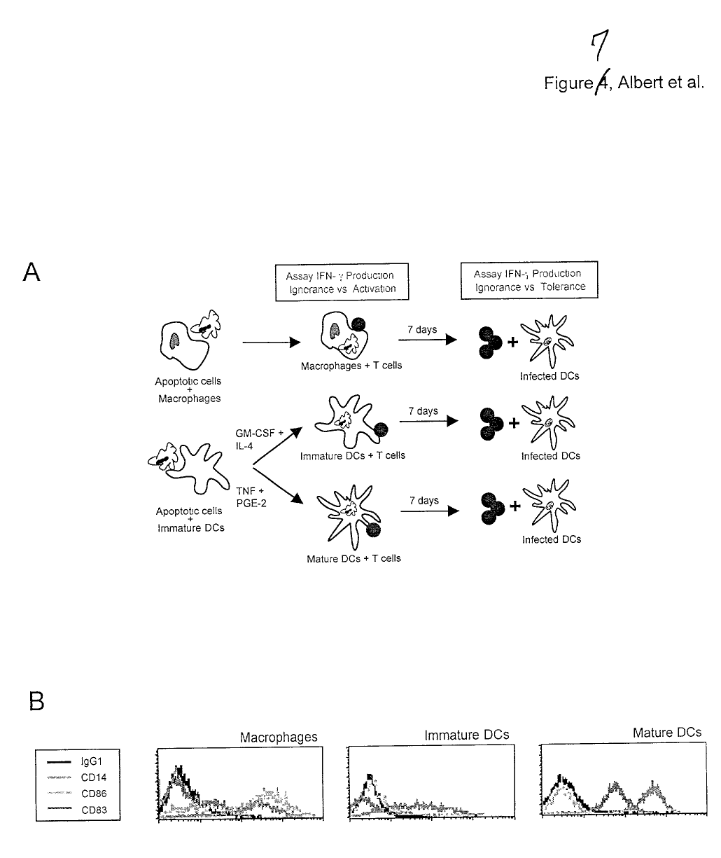

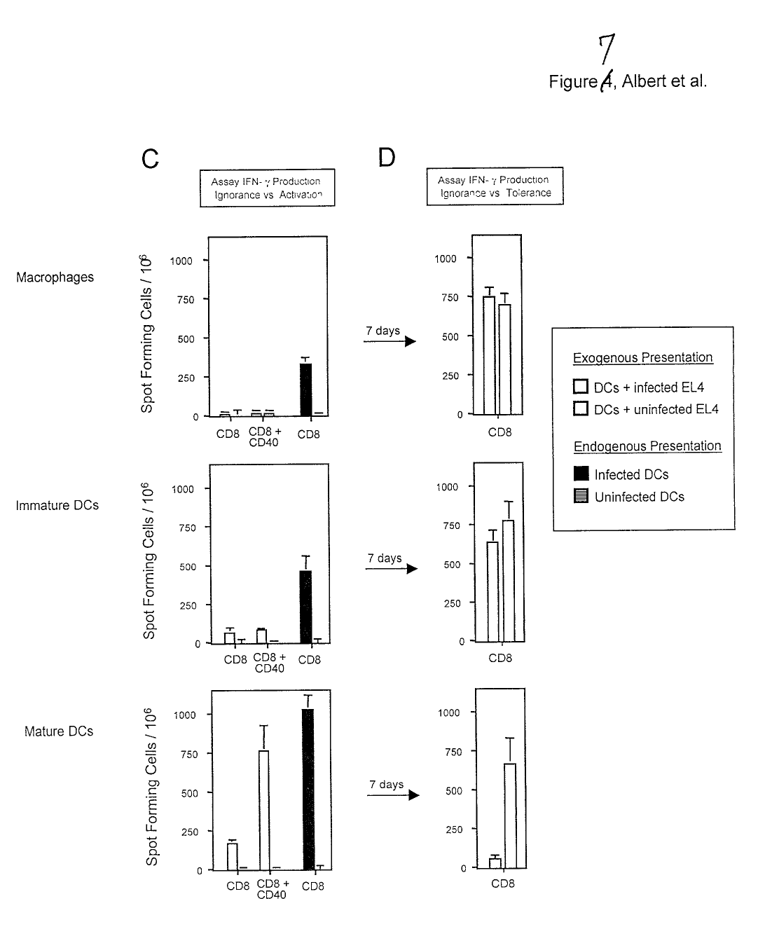

Highly purified CD8⁺ T cells were labeled with the fluorescent dye CFSE and co-cultured for 7 days with DCs that had phagocytosed influenza infected apoptotic EL4 cells. After 3, 5 and 7 days of culture, samples were labeled for HLA-DR (a marker for T cell activation), and for the exposure of phosphatidylserine on the outer leaflet of the plasma membrane using Annexin V (a marker for early apoptosis). Using FACS analysis, the HLA-DR⁺ T cells were gated, and simultaneously evaluated for their CFSE fluorescence and Annexin V staining. On day 3, 12% of the HLA-DR⁺, CD8⁺ T cells had divided and initiated an apoptotic pathway. On day 5, 38% of the dividing HLA-DR⁺, CD8⁺ T cells were Annexin V+. And by day 7, 55% of the proliferating HLA-DR⁺, CD8⁺ T cells had committed to die (FIG. 6). Immature dendritic cells were cocultured with influenza infected apoptotic EL4 cells in the presence of a maturation stimulus as described above. After 36-48 hours, DCs were harvested and cultured with CFSE labeled syngeneic CD8⁺ T cells. After 3, 5 and 7 days, T cells were labeled with HLA-DR-CyChrome and Annexin V-PE and analyzed by FACS. Gating on HLA-DR⁺ T cells allowed for analysis of antigen-reactive T cells (0.8-2 % of the total cell population), permitting the evaluation of Annexin V⁺ cells and relative CFSE fluorescence. With respect to the CFSE intensity, cells were scored based on their mean fluorescence intensity in FL1, thus permitting the determination of how many cell divisions had occurred, and the number of Annexin V⁺ cells in each of these populations. Data is representative of 2 experiments.

By analyzing the relative CFSE intensity, it was demonstrated that most antigen-reactive cells divided 2-4 times prior to initiating a programmed cell death. In CD8⁺ T cell/DC co-cultures exposed to a CD40 stimulus, equivalent levels of dividing HLA-DR⁺ cells could be detected, however insignificant levels of death were observed. Even at day 7, <6% of the proliferating HLA-DR⁺, CD8⁺ T cells were Annexin V⁺. Moreover, it was possible to re-stimulate an influenza-specific T cell response from these T cells (see below). These data indicated that an in vitro strategy had been identified for monitoring the cross-tolerization of CD8⁺ T cells. When CD8⁺ T cells engage a DC cross-presenting antigen in the absence of CD4+T cell help, they divide and are subsequently deleted. Based on in vivo models, it had been assumed that the CD8⁺ T cell proliferation constituted transient activation and that this death was analogous to activation-induced cell death (C. Kurts et al., J Exp Med 186, 2057-62,1997); however these studies demonstrate that while the antigen-responsive dividing cells express ‘activation markers,’ they do not produce IFN-γ and thus should not be considered activated. While T cell tolerance is indeed an active process, it seems to act upstream of T cell stimulation.

The cellular requirements for cross-tolerance were next evaluated and the hypothesis directly tested that resting APCs (e.g. immature DCs) induce tolerance whereas activated APCs (e.g. mature DCs) upregulate costimulatory molecules and thus activate CD8⁺ T cells (S. Gallucci, M. Lolkema, P. Matzinger, Nat Med 5, 1249-55, 1999; D. R. Green, H. M. Beere, Nature 405, 28-9 (2000); K. M. Garza et al., J Exp Med 191, 2021-7, 2000).

As above, immature DCs were cultured with influenza infected apoptotic EL4 cells for 36-48 hours. Either GM-CSF and IL-4, or PGE-2 and TNF-alpha were added to the cultures in order to maintain immature or to generate mature DC populations, respectively. In FIG. 7A, a schematic for the culturing strategy is shown, allowing us to distinguish immunologic ignorance from T cell activation at time=0; and immunologic ignorance from T cell tolerance at time=day 7. Immature DCs were cultured with influenza infected vs. uninfected apoptotic EL4 cells in the presence of either GM-CSF and IL-4, or PGE-2 and TNF-α. In parallel cultures, macrophages from the same donor were cultured with influenza infected apoptotic EL4. In FIG. 7B, upon harvesting the APCs after 36 hours, the cellular phenotype was confirmed by FACS analysis. CD14 is a marker for macrophages which is absent on immature and mature DCs. Surface expression of CD83 is a marker for mature DCs, distinguishing it from immature DCs and macrophages. Additionally, CD80 (B7.1) was also screened on the APC populations to determine the state of activation. In FIG. 7C, After capture of the apoptotic EL4 cells, the different APC populations were co-cultured with syngeneic CD8⁺ T cells in order to assess IFN-y production (A, time=day 0). 6.67×10³ APCs were plated in an ELISPOT well with 2×10⁵ highly purified CD8⁺ T cells +/−agonistic CD40 mAb. Spot forming cells were detected as described in methods. In FIG. 7D, after 7 days of co-culture (A, time=day 7), T cells were collected, cells excluding trypan blue were counted, and plated in fresh wells at a cell dose of 2×10⁵ cells with 6.67×10³ syngeneic influenza infected DCs, thus offering maximal activation to influenza-specific T cells present in the culture. Spot forming cells (SFCs) were detected by ELISPOT as above. In FIG. 7E, to directly test the role for MHC I/TCR and B7/CD28 engagement in cross-tolerance, CD8⁺ T cells were exposed to mature DCs, which had cross-presented influenza antigen, in the presence of W6/32, a blocking mAb specific for HLA-A, B, C; a control IgG1 antibody; or CTLA4-Fc, a soluble fusion protein which binds B7.1 and B7.2, blocking engagement of CD28. Cultures were again tested at time=day 0 in the presence of agonistic CD40 mAb to determine the effect of these blocking agents on T cell activation; and at time=day 7 in the absence of CD40 stimulus in order to determine the effect on cross-tolerance.

In the experiment shown, W6/32 inhibited T cell activation by 95% and completely abrogated the ability to tolerize influenza-specific CD8⁺ T cells. Use of CTLA4-Fc gave a partial phenotype inhibiting T cell activation by 58% and tolerance by 39% in the experiment shown. In all assays (FIGS. 7C-E) SFCs were enumerated in triplicate wells, averaged and plotted as SFC/10⁶ T cells. Error bars indicate standard deviation. Data in FIG. 7 is representative of 3 experiments. NA=Not Applicable.

Additionally, macrophages were tested as an APC capable of cross-tolerizing T cells (FIG. 7A). Upon harvesting the APCs, the maturation phenotype was confirmed by FACS analysis (FIG. 7B). The different APC populations were co-cultured with syngeneic CD8⁺ T cells in order to assess IFN-gamma production using the ELISPOT assay. Immature DCs, apoptotic cells and a DC maturation stimulus (MCM, or a combination of TNF-α and PGE-2) were incubated together for 36-48 hours to allow phagocytosis of the apoptotic EL4 cells, antigen processing and DC maturation to occur. The DCs were collected, counted and added to purified T cell populations in plates precoated with 10 μg/ml of a primary IFN-γ mAb (Mabtech, clone Mab-1-D1K). In all experiments, 2×10⁵T cells were added to 6.67×10³ DCs to give a 30:1 DC:T cell ratio. The cultures were incubated in the plates for 40-44 hours at 37 ° C. At that time, cells were washed out using mild detergent and the wells were then incubated with 1 μg/ml biotin-conjugated IFN-γ mAb (Mabtech, clone Mab 7BG-1). Wells were next stained using the Vectastain Elite kit as per manufacturers instructions (Vector Laboratories). Colored spots represented the IFN-γ releasing cells and are reported as spot forming cells/10⁶ cells. Triplicate wells were averaged and means reported.

In parallel wells, cultures were incubated for 7 days and T cells were tested for the ability to recall an influenza-specific immune response (FIG. 7A). If the antigen-reactive T cells were being tolerized by a deletional mechanism as indicated by data in FIG. 6, the influenza-specific T cells should no longer be present at day 7.

As alluded to above, the absence of CD4⁺ T cell help prevented the CD8⁺ T cells from producing significant IFN-γ when stimulated with DCs loaded with antigen via the exogenous pathway (FIG. 7C). When mature DCs were co-cultured in the presence of agonistic CD40 mAb, it was possible to generate a response equivalent to that achieved using mature DCs presenting antigen via the endogenous pathway (FIG. 7C). Immature DCs were not able to stimulate IFN-γ production even in the presence of agonistic CD40 mAb (FIG. 7C). While immature DCs are capable of cross-presenting antigen and generating surface MHC I/peptide complexes [M. L. Albert et al., J Exp Med 188, 1359-68 (1998)], CD40 stimulation is not sufficient to permit T cell activation. This is likely due to low CD40 expression on immature DCs. Macrophages cannot cross-present antigen [M. L. Albert et al., J Exp Med 188, 1359-68 (1998)], confirmed here by demonstrating their inability to stimulate a CD8⁺ T cell response via the exogenous pathway (FIG. 7C). Comparing the ability of each APC population to activate T cells via the endogenous vs. exogenous MHC I presentation pathways demonstrates the integrity of each cell type. This data also illustrates that it is not possible to make a quantitative comparison of the three APC populations—stimulatory capacity is likely due to higher levels of MHC I and costimulatory molecules on mature DCs as compared to immature DCs and macrophages. To examine the proliferative ability of CD8⁺ T cells in response to the different APC populations, parallel cultures were exposed to ³H-Thymidine on day 4.5 and cellular proliferation was determined. As in FIG. 5B, the CD8⁺ T cells exposed to mature DCs charged with antigen via the exogenous pathway proliferated to the same extent as CD8⁺ T cells cultured in the presence of agonistic CD40 mAb. Only minimal proliferation was detected in cultures of CD8⁺ T cells exposed to immature DCs or macrophages co-cultured with influenza infected apoptotic EL4 cells.

Distinguishability between T cell ignorance and T cell tolerance in CD8⁺ T cells exposed to the different APC populations was then tested (FIG. 7A). In the former influenza-responsive cells persist, as there is no antigen-specific engagement between the APC and the T cells; whereas in the latter, the influenza-specific T cells are actively deleted and cannot be recalled. After 7 days in co-culture, T cells were collected; cells excluding trypan blue were counted; and the T cells were plated in fresh wells with syngeneic influenza infected DCs (T:DC ratio 30:1), thus offering maximal activation to influenza-specific T cells present in the culture. In 3/3 independent experiments, no IFN-γ production could be detected in the population of CD8⁺ T cells which had been exposed to mature DCs cross-presenting influenza antigen (FIG. 7D). It was therefore concluded that the influenza-specific T cells had been deleted as suggested by FIG. 3. In contrast, if uninfected EL4 cells were used as a source of apoptotic cells, the CD8⁺ T cells did not proliferate (FIG. 5B), and when these T cells were removed from the co-culture and stimulated with influenza infected DCs, influenza-reactive T cells could be detected (FIG. 7D). This data suggests that the influenza-specific CD8⁺ T cells in these cultures remained immunologically ignorant during the 7 days of co-culture. Strikingly, CD8⁺ T cells exposed to immature DCs that had captured influenza infected apoptotic cells displayed a phenotype consistent with immunologic ignorance. This was evident by the ability to recall an influenza-specific T cell response upon maximal stimulation (FIGS. 7A and 7D).

The current ‘two signal’ model for T cell activation vs. tolerance proposes that in the absence of costimulatory molecular interactions, such as B7-1 or B7-2, TCR engagement results in tolerance induction [S. Guerder, R. A. Flavell, Int Rev Immunol 13, 135-46 (1995); J. G. Johnson, M. K. Jenkins, Immunol Res 12, 48-64 (1993)]. According to this model, a maturation stimulus for immature dendritic cells, possibly offered by a ‘danger signal,’ is what distinguishes priming vs. tolerance [S. Gallucci, M. Lolkema, P. Matzinger, Nat Med 5, 1249-55 (1999); J. M. Austyn, Nat Med 5, 1232-3 (1999)]. To directly test this hypothesis, CD8⁺ T cells were exposed to mature DCs, which had cross-presented influenza antigen, in the presence of: W6/32, a blocking mAb specific for HLA-A, B, C; or CTLA4-Fc, a soluble fusion protein which binds B7.1 and B7.2, blocking engagement of CD28. In the presence of W6/32, T cell activation was inhibited (FIG. 7E), as was proliferation at day 4.5. Without engagement of the TCR, or ‘signal 1,’ the T cells were neither activated, nor were they tolerized, as evident by the ability to recall an influenza-specific immune response after 7 days of culture (FIG. 7E). Inhibition with CTLA4-Fc gave a partial phenotype: 45-60% inhibition T cell activation (FIG. 7E); 30-50% inhibition of proliferation at day 4.5; and 40-50% inhibition of tolerance induction (FIG. 7E).

These data demonstrate that cross-tolerance is an active process which results in deletion of antigen-specific CD8⁺ T cells; that DC maturation is required for cross-tolerance of CD8⁺ T cells; and that multiple co-stimulatory molecules (e.g. ICAM-1, HSA and LFA-3) are likely to be important for efficient tolerization of antigen-specific CD8⁺ T cells. Contrary to what has been proposed, these data argue that the same CD83⁺ myeloid-derived mature DC is capable of both activating and tolerizing antigen-specific CD8⁺ T cells.

The foregoing data indicates that the bone marrow derived cell responsible for mediating cross-tolerance is the dendritic cell, and that antigen transfer for cross-tolerization is achieved by phagocytosis of apoptotic material, thus permitting access to MHC I. These findings are supported by the observation that increased apoptotic death increases the efficiency of cross-tolerance (6), and that DCs are the only APC capable of capturing antigen in the periphery and entering the draining lymphatics [J. Banchereau, R. M. Steinman, Nature 392, 245-52 (1998)]. An unexpected result borne from our studies challenges a major paradigm in the field of immunobiology. To achieve cross-tolerance, DC maturation is required. The critical checkpoint does not appear to be a maturation stimulus as suggested by the two signal hypothesis, but is instead the presence of CD4⁺ helper T cells, which act in part by delivering a signal to the mature DC via CD40. Again, in considering the physiologic relevance of this finding, it is intriguing to take into account the requirements for DCs to reach the T cell zone of draining lymph organs. Only mature DCs seem capable of accessing the T cells in lymph organs as expression of the chemokine receptor CCR7 (expressed on mature but not immature DCs) is critical for T cell/DC colocalization (24).

### EXAMPLE 3

### Abrogation of Effective CD4⁺ Help by Interfering with Signal Transduction Events in the DC Post-CD4/DC Interaction

The cross-presentation of tissue-restricted antigen can be modeled in vitro as a two step process. First, immature dendritic cells are incubated with apoptotic cells in the presence of TNF-alpha and PGE-2, resulting in antigen capture and maturation. After 36 hours, the DCs are harvested and co-cultured with bulk T cells in order to determine the immunologic outcome—CTL activation vs. tolerization. In a screen for compounds which act on the DC to inhibit cross-priming, it was discovered unexpectedly that the immunophilin FK506 acts downstream of CD40 and prevents the DC from activating antigen-specific CD8+ T cells. Notably, this effect is independent of its action on T cells. As will be seen below, it has been confirmed that FK506 does not affect the DC's ability to phagocytose the apoptotic cell; nor does this compound influence DC maturation. In fact, MHC I/peptide complexes are still generated in the presence of this inhibitor, however instead of T cell activation, the CTLs are actively tolerized. Surprisingly, a closely related molecule, Cyclosporin A (CsA), does not inhibit the cross-priming of CTLs via the apoptosis-dependent MHC I antigen presentation pathway. CsA is known to bind a family of cyclophilins, allowing for the binding of calcineurin. FK506 binds FKBPs (including FKBP 12) and in turn forms a complex with calcineurin. Taken together, this data supports a role for FKBPs in skewing cross-presentation towards tolerance, which is independent of calcineurin. The work herein has shown that FK506 can block CD40 signaling and can therefore skew the cross-presentation of apoptotic material towards cross-tolerization of CTLs.

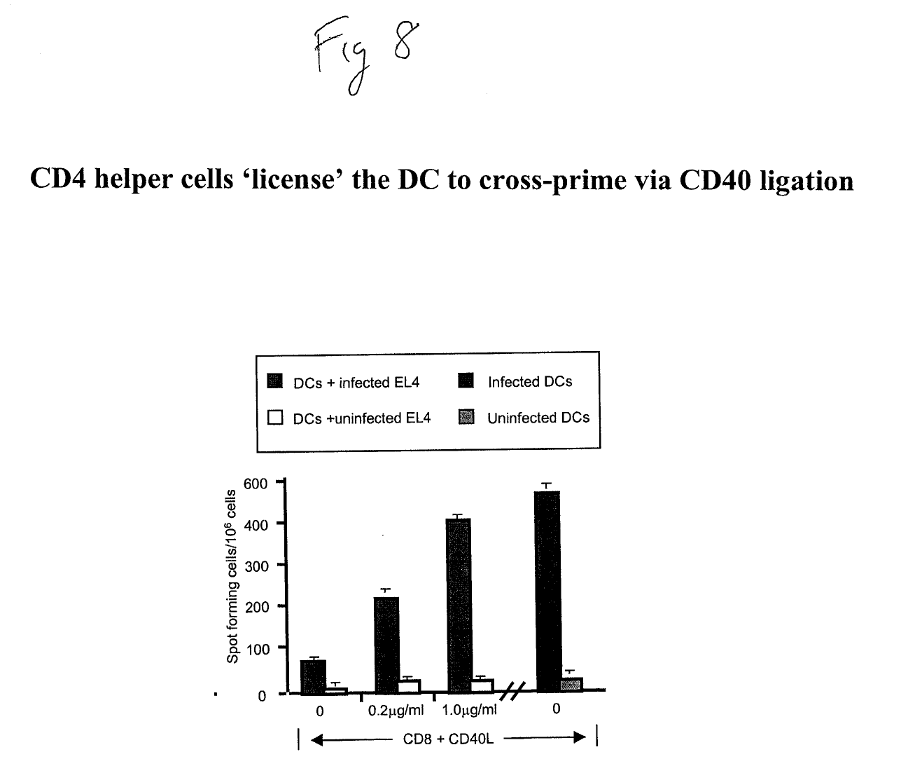

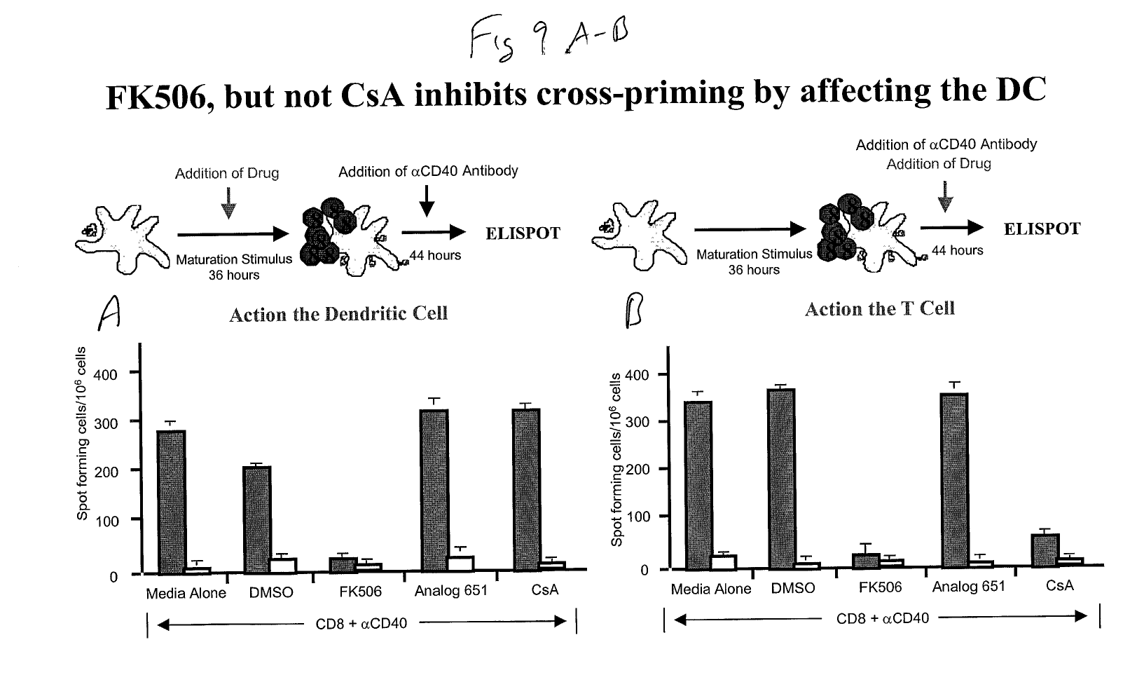

CD40L is able to substitute for CD4+T-cell help in the cross-priming of CD8+ T cells. FIG. 8 shows a dose-response effect of CD40L in substituting for CD4+ help in cross-priming CD8+ T cells. As in FIGS. 2 and 4, apoptotic cells expressing influenza antigen can be cross-presented by DCs for the activation of CD8⁺ T cells if and only if CD4⁺ T cells or a substituting agent such as CD40L is present in the co-cultures.

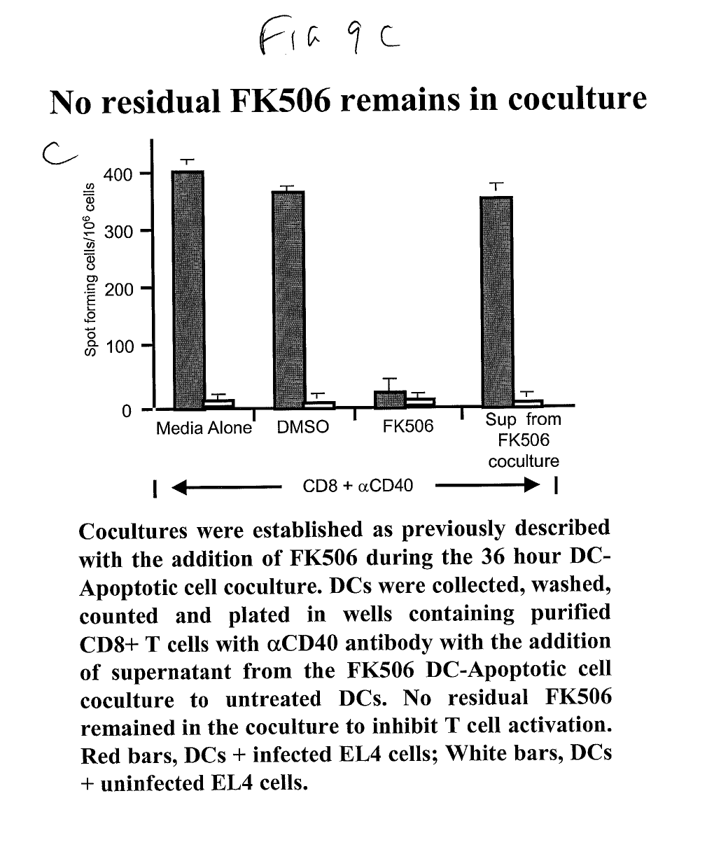

FIGS. 9A-C shows that FK506, but not cyclosporin nor analog 651 (an FK506analog which possesses an FKBP binding domain but no calcineurin binding domain), inhibits cross-priming by affecting the dendritic cells. EL4 cells are infected with influenza and allowed to express influenza proteins for 5 hours. The cells are then UVB irradiated and allowed to undergo apoptosis for 8 hours. At this time, day 6 immature DCs are added in the presence of a maturation stimulus (TNF-alpha and PGE-2), +/−the addition of various immunophilins. After 0 36 hours mature DCs are harvested and plated in wells containing purified CD8+ T cells with agonistic anti-CD40 mAb.

As evident by the abrogation of IFN-gamma, FK506 is capable of blocking the dendritic cells ability to activate T cells via the exogenous pathway (FIG. 9A).

The FK506 and CsA were also placed into culture at the time of co-culture with T cells, thus directly effecting the signal transduction of the T cells in preventing calcineurin-mediated T cell activation. Expectedly, CsA and FK506 both inhibited T cell activation through its effect on calcineurin (FIG. 9B).

This however is not the mechanism by which the FK506 is blocking the activation of T cells via the cross-presentation pathway, as residual drug is removed prior to the DCs being added to the T cells (FIG. 9C) co-cultureco-cultureNo residual FK506 remained in the co-culture to inhibit T cell activation (FIG. 9C). Dark bars, DCs+infected EL4 cells; White bars, DCs+uninfected EL4 cells.

Similar data was obtained using Rapamycin, an inhibitor of TOR.

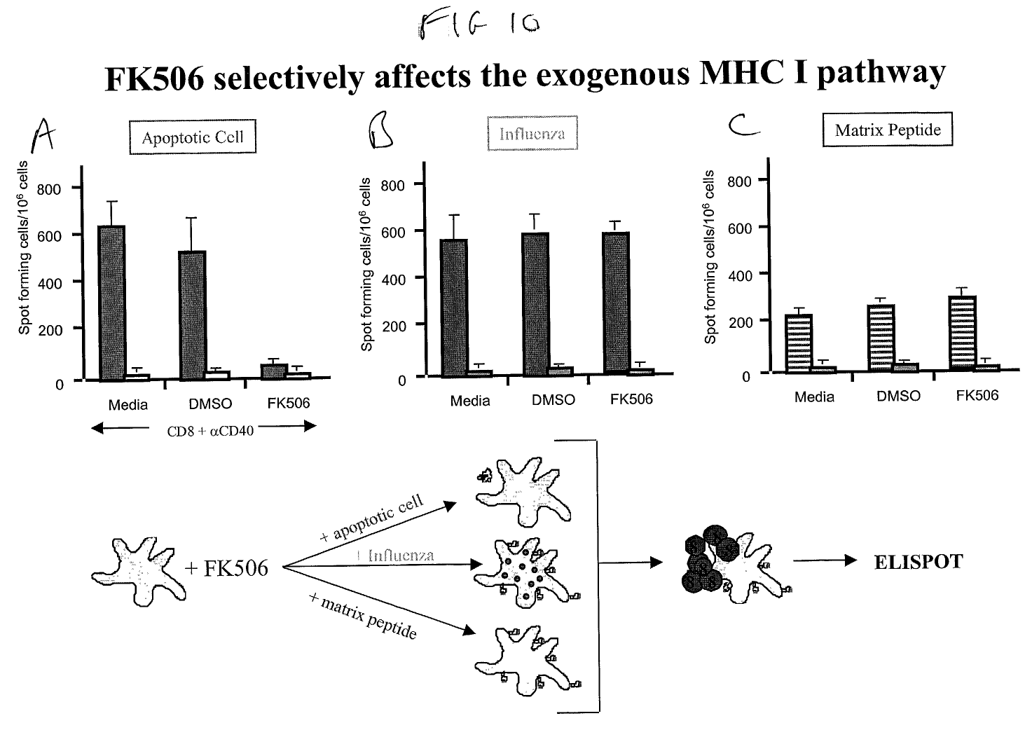

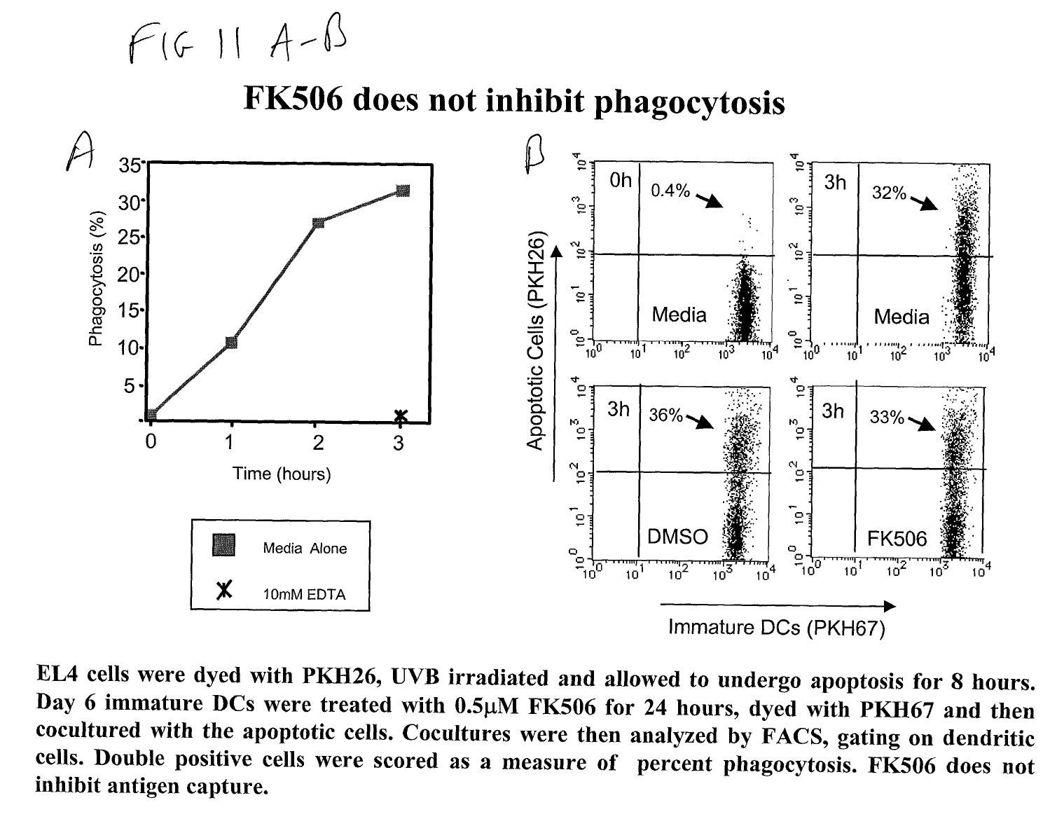

FIG. 10 shows that FK506 selectively affects the exogenous MHC I pathway. Using designs similar to the foregoing, with antigen presented by the exogenous pathway (left panel) using an apoptotic cell, the endogenous pathway (influenza, center panel), or by simply surface loading MHC I using soluble matrix peptide (right panel), the ability of FK506 to abrogate activation of T cells by only the exogenous route is demonstrated. Note, this data also confirms that the FK506 is not directly acting on the T cell. Similar data has been achieved using Rapamycin. Cocultures were established as previously described. Parallel A2.1+ DCs were matured and treated with 0.5 uM FK506. Upon co-culture with purified CD8+ T cells, these various DC groups were directly infected with influenza or pulsed with A2.1 restricted matrix peptide. ELISPOT assay was performed and spot forming cells/10⁶ cells are reported. While FK506 can inhibit T cell activation in the exogenous pathway, no effect is seen on DCs directly infected with live virus endogenously presenting to T cells or DCs pulsed with peptide activating CD8+ T cells. Red bars, DCs+infected EL4; white bars, DCs+uninfected EL4; Black bars, infected DCs; gray bars, uninfected DCs; Striped bars, peptide pulsed DCs; gray bars, unpulsed DCs.

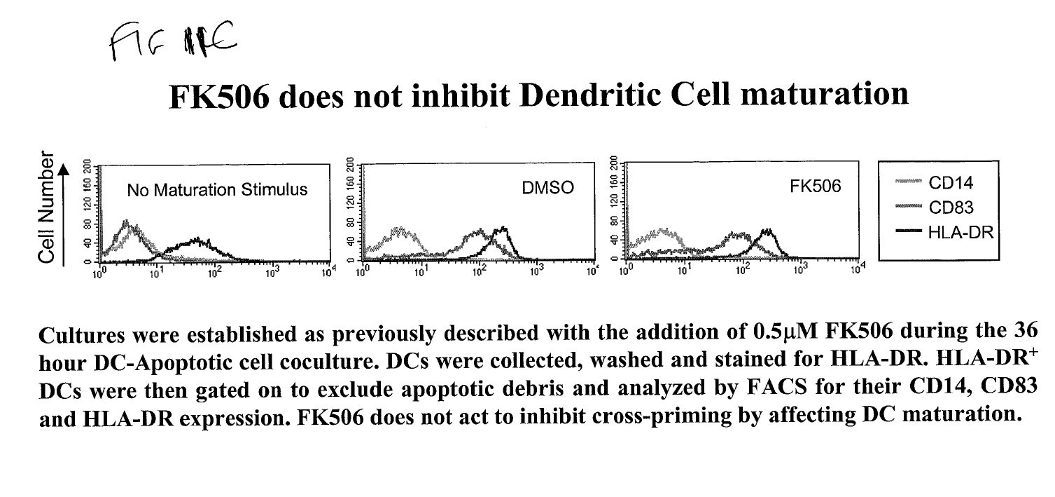

To determine the mechanism of FK506-mediated inhibition of cross-presentation, we first asked if the apoptotic material was being captured and cross-presented by the maturing DC. FIGS. 11A-C shows that FK506 in fact does not inhibit phagocytosis, dendritic cell maturation or the generation of MHC I/peptide complex. EL4 cells were dyed with PKH26, UVB irradiated and allowed to undergo apoptosis for 8 hours. Day 6 immature DCs were treated with 0.5 micromolar FK506 for 24 hours, dyed with PKH67 and then co-cultured with the apoptotic cells. Co-cultures were then analyzed by FACS, gating on dendritic cells. Double positive cells were scored as a measure of percent phagocytosis. FK506 does not inhibit antigen capture (FIG. 11A).

FIG. 11B shows that FK506 does not inhibit dendritic cell maturation. Cultures were established as previously described with the addition of 0.5 micromolar FK506 during the 36 hour DC-Apoptotic cell co-culture. DCs were collected, washed and stained for HLA-DR. HL-ADR+DCs were then gated on to exclude apoptotic debris and analyzed by FACS for their CD14, CD83 and HLA-DR expression. FK506 does not act to inhibit activation of T cells via the exogenous pathway by affecting DC maturation.

FIG. 1C shows that FK506 does not inhibit generation of MHC I/peptide complexes. Dendritic cells cross-presenting influenza antigen derived from apoptotic cells were loaded with chromium and subjected to influenza-specific CTLs. If the DCs are effective targets, it indicates that they have generated MHC I/peptide complexes where the peptide was derived from the exogenous antigen. By demonstrating that FK506 treated DCs cross-presenting antigen derived from apoptotic cells can indeed serve as targets for influenza-specific CTLs we show that FK506 does not inhibit generation of MHC I/peptide complexes via this exogenous pathway.

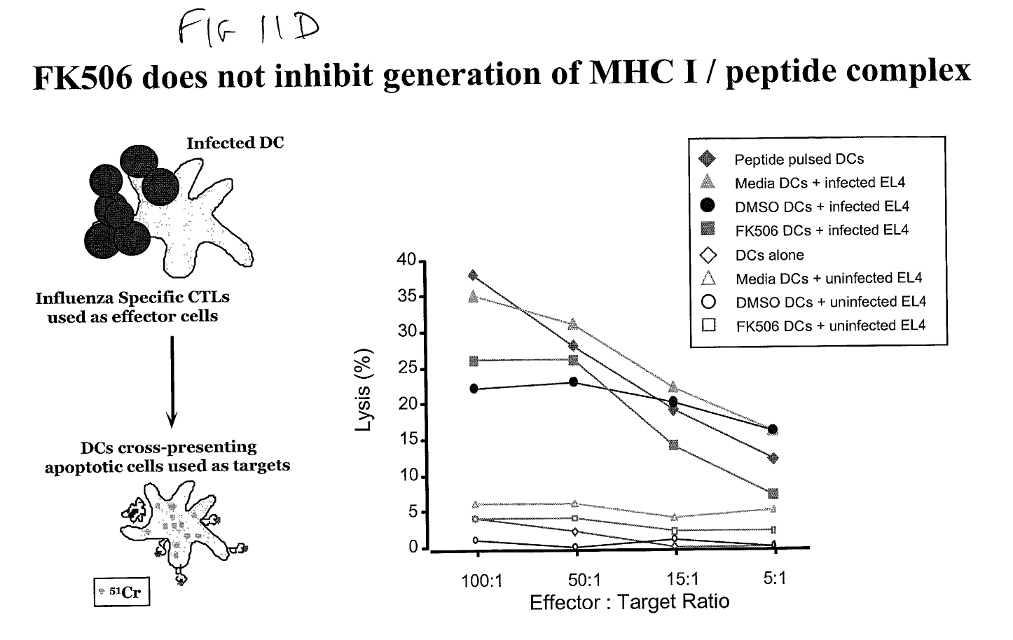

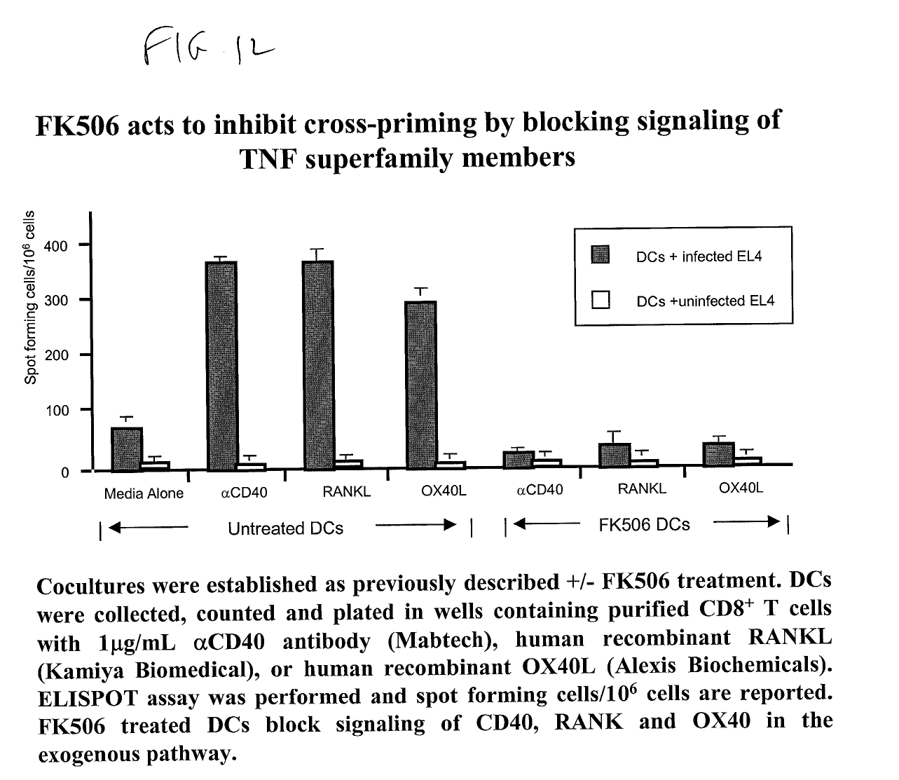

Instead, we find that FK506 inhibits the DC from receiving CD40 help. FIG. 12 shows that FK506 acts to inhibit activation of T cells via the exogenous pathway by blocking the signaling of TNF superfamily members. Co-cultures were established as previously described +/−FK506 treatment. DCs were collected, counted and plated in wells containing purified CD8+ T cells with Imicrog/mL anti-CD40 antibody (Mabtech), human recombinant RANKL (Kamiya Biomedical), or human recombinant OX40L (Alexis Biochemicals). ELISPOT assay was performed and spot forming cells/10⁶ cells are reported. FK506 treated DCs block signaling of CD40, RANK and OX40 in the exogenous pathway and prevent the release of IFN-γ from antigen-specific T cells. Similar results have been obtained with Rapamycin.

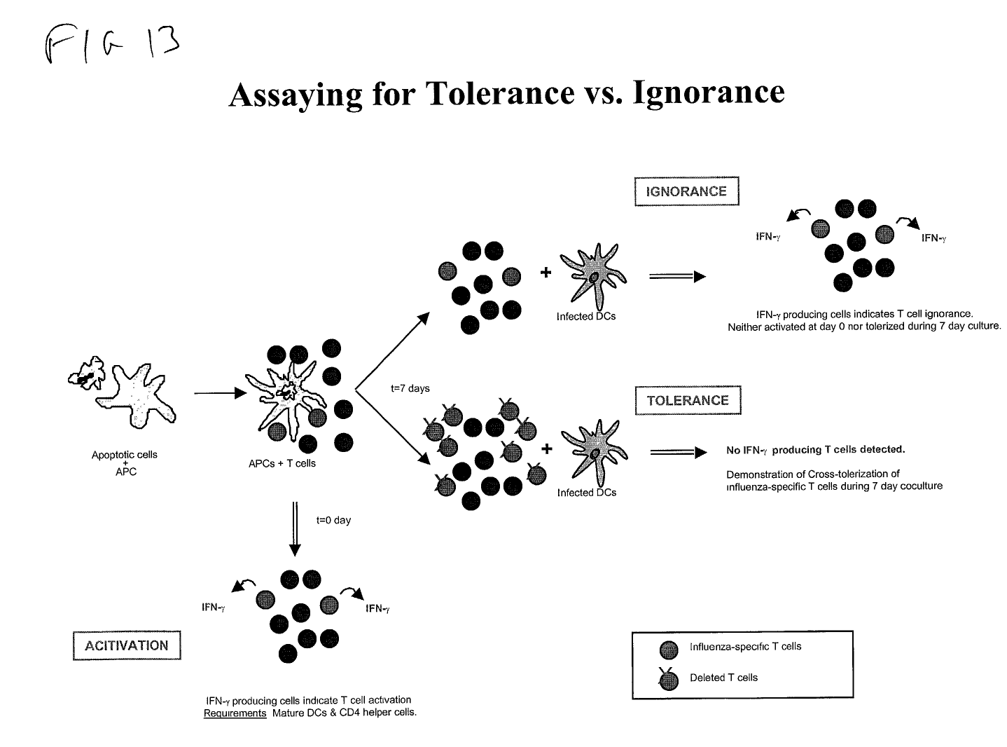

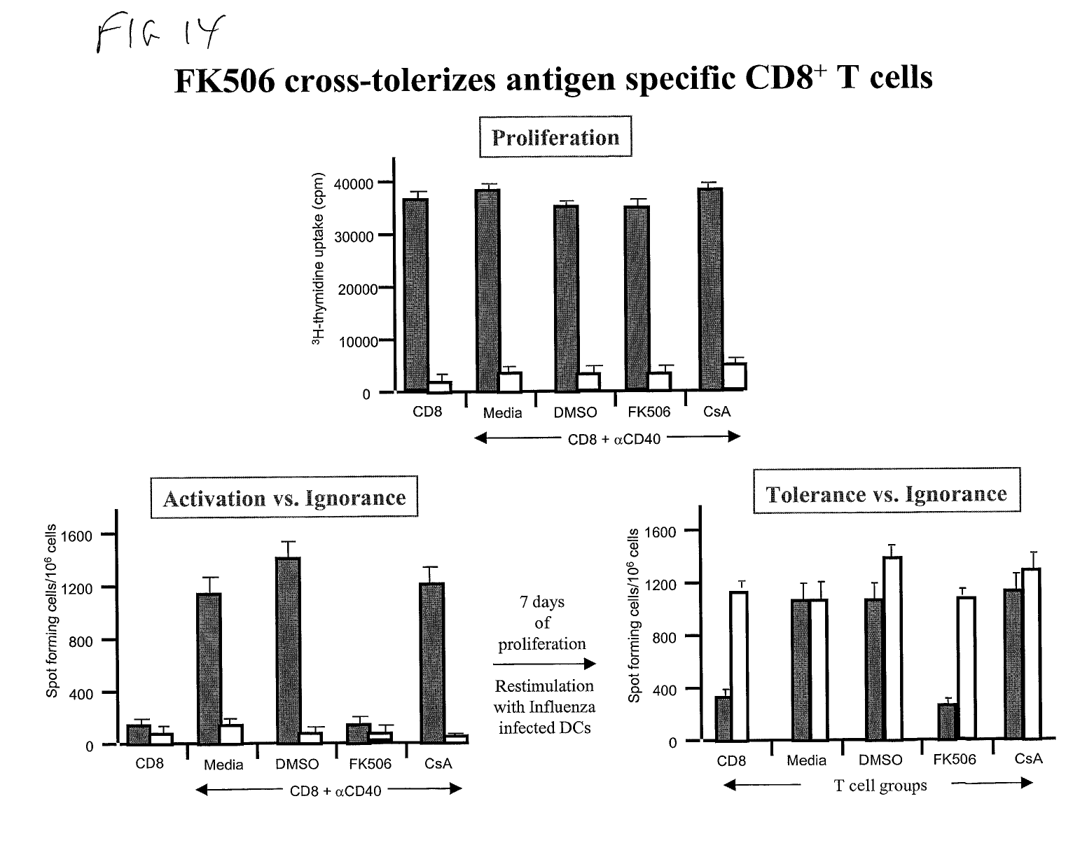

FIG. 13 shows the procedure used to assay for tolerance versus ignorance. Using this assay, and the foregoing materials and methods, FIG. 14 shows that FK506 cross-tolerizes antigen-specific CD8+ T cells. Co-cultures were established as previously described. DCs were collected, washed, counted and plated with purified CD8+T cells (+/−αCD40 antibody) and ELISPOT assay was performed. The DC-T cell co-cultures were allowed to proliferate for 5 days and assayed for 3H-thymidine uptake. At 7 days of co-culture, T cells were then collected, counted and plated in wells containing syngeneic DCs directly infected with influenza. ELISPOT assay was performed to assess tolerance vs. ignorance. CD8⁺ T cells co-cultured with FK506 treated DCs cross-presenting influenza antigen proliferate but do not release IFN-γ, as do CD8⁺ T cells that have not received CD4 help. When these proliferating CD8⁺ T cells are restimulated with influenza infected DCs (providing maximal stimulation), they still do not release IFN-γ suggesting that they have been tolerized. This is in contrast to CD8⁺ T cells co-cultured with DCs fed with uninfected EL4 cells, which remain immunologically ignorant and are able to release IFN-γ upon maximal restimulation with influenza infected DCs.

The foregoing results demonstrate that FK506 possesses heretofore unappreciated immunosuppressive effects which may be used in the practice of the methods described herein. As shown in the foregoing studies, FK506 blocks CD40 signalling to skew cross-presentation towards cross-tolerizing of CTLs. CD4+ T cells ‘license’ the dendritic cells to cross-prime CD8+ T cells via CD40 ligation. FK506 acts to inhibit cross-priming by blocking CD40 signaling and signaling of other TNF superfamily members. FK506 skews the cross-presentation of apoptotic material towards the cross-tolerization of CTLs. This finding is exploited in the ex-vivo and in-vivo methods of the invention, described above.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A method for inducing tolerance in a mammal to a pre-selected antigen comprising the steps of
a. isolating peripheral blood mononuclear cells (PBMC) from a whole blood sample from said mammal;
b. isolating dendritic cells from said PBMC;
c. exposing said dendritic cells ex vivo to apoptotic cells expressing said pre-selected antigen in the presence of at least one dendritic cell maturation stimulatory molecule and in the absence of effective CD4+ T cell help;
d. introducing a cellular portion of step c) into said mammal;
wherein said dendritic cells induce apoptosis of antigen-specific CD8+ T cells in said mammal resulting in tolerance to said antigen.

2. The method of claim 1 wherein said dendritic cell maturation stimulatory molecule is PGE2, TNF-alpha, lipopolysaccharide, monocyte conditioned medium, CpG-DNA, or any combination thereof.

3. The method of claim 1 wherein said absence of effective CD4+ T cell is achieved by excluding CD4+ T cells from said step c).

4. The method of claim 1 wherein said absence of effective CD4+ T cell help is achieved by including in step c) at least one agent that inhibits or eliminates effective CD4+ T cell help.

5. The method of claim 4 wherein said agent which inhibits or eliminates effective CD4+ help is a monoclonal antibody to a TNF superfamily member, a combination thereof, a monoclonal antibody to a receptor for a TNF superfamily member, or a combination thereof

6. The method of claim 5 wherein said TNF superfamily member is CD40L, TRANCE, OX40 or DR3.

7. The method of claim 5 wherein said receptor for a TNF superfamily member is CD40, TRANCE, OX40 ligand or TWEAK.

8. The method of claim 1 wherein said absence of effective CD4+ T cell is achieved by inhibiting formation of mature forms of MHC II/peptide complexes within the dendritic cell.

9. The method of claim 8 wherein said inhibiting is achieved by preventing cleavage of invariant chain.

10. The method of claim 9 wherein said preventing is achieved by addition of a cathepsin inhibitors.

11. The method of claim 8 wherein said inhibiting is achieved by blocking loading of peptides by inhibiting HLA-DM.

12. The method of claim 8 wherein said inhibiting is achieved by preventing successful antigen degradation and formation of a MHC II peptide epitope.

13. The method of claim 12 wherein said preventing is achieved by inhibiting cathepsin D or alternative proteases.

14. The method of claim 8 wherein said inhibiting is achieved by inhibiting transport of MHC II/peptide complexes to the cells surface.

15. The method of claim 4 wherein said agent which inhibits or eliminates effective CD4 T cell help inhibits signalling consequent to dendritic cell-CD4 T cell engagement.

16. The method of claim 15 wherein said agent is selected from a FKBP antagonist and a TOR antagonist.

17. The method of claim 16 wherein said FKBP antagonist is FK-506.

18. The method of claim 16 wherein said TOR antagonist is rapamycin.

19. The method of claim 1 wherein said pre-selected antigen is a tumor antigen, a viral antigen, a self antigen or a transplant antigen.

20. The method of claim 4 wherein said presence of at least one agent that inhibits effective CD4 T cell help comprises a monoclonal antibody to TRANCE and FK-506.

21. The method of claim 1 wherein after a period of time following step c), a cellular portion is infused into the mammal.

22. The method of claim 1 wherein said mammal is a human.

23. A method for inducing tolerance in a mammal to a pre-selected antigen comprising the steps of
a. providing a dendritic cell chemoattractant at a site in a mammalian body, said site comprising an antigen to which tolerization of an immune response is desired or made to comprise an antigen to which tolerization of an immune response is desired by administration of said antigen to said site; and
b. administering to said site or systemically to said mammal an agent which inhibits or eliminates effective CD4+ T cell help;
wherein immune system cells of said mammal are tolerized to said antigen.

24. The method of claim 23 wherein said dendritic cell chemoattractant is a ligand for CCR6.

25. The method of claim 23 wherein said ligand for CCR6 is 6-C-kine.

26. The method of claim 23 wherein said agent which inhibits or eliminates effective CD4+ help is a monoclonal antibody to a TNF superfamily member, a combination thereof, a monoclonal antibody to a receptor for a TNF superfamily member, or a combination thereof.

27. The method of claim 26 wherein said TNF superfamily member is CD40L, TRANCE, OX40 or DR3.

28. The method of claim 26 wherein said receptor for a TNF superfamily member is CD40, TRANCE, OX40 ligand or TWEAK.

29. The method of claim 23 wherein said agent which inhibits or eliminates effective CD4+ T cell inhibits formation of mature forms of MHC II/peptide complexes within the dendritic cell.

30. The method of claim 29 wherein said inhibits formation is achieved by preventing cleavage of invariant chain.

31. The method of claim 29 wherein said inhibits or eliminates is achieved by addition of a cathepsin inhibitor.

32. The method of claim 29 wherein said inhibiting is achieved by blocking loading of peptides by inhibiting HLA-DM.

33. The method of claim 32 wherein said inhibiting is achieved by preventing successful antigen degradation and formation of a MHC II peptide epitope.

34. The method of claim 33 wherein said preventing is achieved by inhibiting cathepsin D or alternative proteases.

35. The method of claim 29 wherein said inhibiting is achieved by inhibiting transport of MHC II/peptide complexes to the cells surface.

36. The method of claim 23 wherein said agent which inhibits or eliminates effective CD4 T cell help inhibits signalling consequent to dendritic cell-CD4 T cell engagement.

37. The method of claim 36 wherein said agent is selected from a FKBP antagonist and a TOR antagonist.

38. The method of claim 37 wherein said FKBP antagonist is FK-506.

39. The method of claim 37 wherein said TOR antagonist is rapamycin.

40. The method of claim 23 wherein said pre-selected antigen is a tumor antigen, a viral antigen, a self antigen or a transplant antigen.

41. The method of claim 23 wherein said presence of at least one agent that inhibits effective CD4 T cell help comprises a monoclonal antibody to TRANCE and FK-506.
